(19) United States
(12) Patent Application Publication
(10) Pub. No.: US 2022/0298493 A1
(43) Pub. Date: Sep. 22, 2022

(54) INACTIVATION PROCESS FOR VIRUSES

(71) Applicant: Valneva Austria GmbH, Vienna (AT)

(72) Inventors: Robert Schlegl, Siegenfeld (AT); Jürgen Heindl-Wruss, Vienna (AT)

(73) Assignee: Valneva Austria GmbH, Vienna (AT)

(21) Appl. No.: 17/688,960

(22) Filed: Mar. 8, 2022

Related U.S. Application Data
(63) Continuation of application No. PCT/EP2020/075223, filed on Sep. 9, 2020.

(30) Foreign Application Priority Data
Sep. 9, 2019 (EP) 19196192.9

(51) Int. Cl. C12N 7/00

(52) U.S. Cl. CPC C12N 7/00; C12N 2770/24163; C12N 2770/36163

(57) ABSTRACT

Described herein are methods for inactivation of viruses with high yield and recovery, and compositions produced by such methods.

### RELATED APPLICATION

This application is a continuation of International Patent Application Serial No. PCT/EP2020/075223, filed Sep. 9, 2020, the entire contents of which is incorporated by reference herein in its entirety.

### FIELD

The disclosure relates to processes for the inactivation of viruses with improved recovery.

### BACKGROUND

In the production of inactivated viral vaccines, one of the most crucial steps is inactivation of the infectious virus particles. Chemical inactivation of viruses must be conducted for a sufficient time and with a sufficient amount or concentration of chemical inactivation agent to fully inactivate the virus while retaining functional epitopes important for the induction of protective immunity. For some commercial vaccines, such as IXIARO®, a Japanese encephalitis virus (JEV) vaccine, the inactivation is done by incubation of the purified active virus material with formaldehyde for a defined period of time such as 10 days.

### SUMMARY

During and after inactivation of JEV, a precipitate was observed, which was determined to include JEV virus, resulting in a loss of yield of inactivated virus. The causes for this precipitate were investigated. Unexpectedly, the loss due to precipitation was determined to result from inactivation process parameters, including mechanical stress resulting from agitation of the inactivation mixture. In addition, the majority of the loss unexpectedly was found to occur in the first few hours of incubation of JEV with formaldehyde.

Aspects of the invention provide the following:

A1. A method of inactivating a virus (or viruses) comprising

contacting a liquid composition comprising the virus(es) with a chemical viral inactivating agent in a container,

mixing the chemical viral inactivating agent and the liquid composition comprising the virus(es) under conditions of laminar flow but not turbulent flow, and incubating the chemical viral inactivating agent and the liquid composition comprising the virus(es) for a time sufficient to inactivate the virus(es).

A2. The method of aspect A1, wherein mixing of the chemical viral inactivating agent and the liquid composition comprising the virus(es) is performed in a flexible bioreactor or single use bioprocess bag.
A3. The method of aspect A2, wherein the mixing is performed under conditions that result in a modified Reynolds Number (Re_{mod}) of less than 1000, as determined by formula (1):

Re_{mod}=((V*k*C*D))/(15*v*(2*h+B)) (1),

wherein V is the volume of the flexible bioreactor bag, k is the mixing (rocking) rate of the flexible bioreactor bag, C and D are correlation factors determined for the flexible bioreactor bag, v is the kinematic viscosity of the liquid in the flexible bioreactor bag, h is the height of liquid in flexible bioreactor bag, and B is the width of the flexible bioreactor bag.

A4. The method of any one of aspects A1-A3, wherein the mixing comprises inverting the container not more than 1, 2, 3, 4 or 5 times during the period of incubation.
A5. The method of any one of aspects A1-A3, wherein the mixing comprises subjecting the container to rocking, rotation, orbital shaking, or oscillation for not more than 15 seconds, 30 seconds, 1 minute, 2 minutes, 5 minutes, or 10 minutes at not more than 2 rpm, 5 rpm, or 10 rpm, during the period of incubation.
A6. The method of any one of aspects A1-A5, wherein the mixing is performed only within the first 10 minutes, 20 minutes, 30 minutes, 40 minutes, 50 minutes, or 60 minutes after the contacting of the viruses and the agent in the container, or wherein no mixing is performed after 2 hours, 4 hours, 8 hours, 12 hours, 24 hours, or 48 hours from the contacting of the viruses and the agent in the container.
A7. The method of any one of aspects A1-A6, wherein the inactivation of the virus is completed in a time period that is not more than 10% longer than the time period for inactivation of the same virus(es) using the same chemical viral inactivation agent without any restriction on mixing.
A8. The method of any one of aspects A1-A7, wherein the chemical viral inactivation agent comprises or consists of formaldehyde; enzyme; β-propiolactone; ethanol; trifluroacetic acid; acetonitrile; bleach; urea; guanidine hydrochloride; tri-n-butyl phosphate; ethylene-imine or a derivative thereof; an organic solvent, optionally Tween, Triton, sodium deoxycholate, or sulfobetaine; or a combination thereof.
A8.1. The method of any one of aspects A1-A7, wherein the inactivation of the viruses is done by low pH treatment, e.g. for the production of monoclonal antibodies in order to inactivate viruses.
A9. The method of any one of aspects A1-A8, wherein the chemical viral inactivating agent and the liquid composition comprising viruses are incubated for 1-20 days.
A10. The vaccine of any one of aspects A1-A9, wherein the chemical viral inactivating agent and the liquid composition comprising virus are incubated at about 10° C. to about 30° C.
A11. The method of any one of aspects A1-A10, wherein the virus is a RNA virus.
A12. The method of aspect A11, wherein the RNA virus belongs to a virus family selected from the group consisting of Flaviviridae, Togaviridae, Paramyxoviridae, Picornaviridae, Orthomyxoviridae, Filoviridae, Arenaviridae, Rhabdoviridae, and Coronaviridae.
A13. The method of aspect A12, wherein the virus is selected from the group consisting of Japanese encephalitis virus, Zika virus, Yellow Fever virus, Dengue virus, thick born encephalitis virus, polio virus, hepatitis A virus, rabies virus, hepatitis B virus, hepatitis C virus and Chikungunya virus.
A14. The method of any one of aspects A1-A13, wherein the liquid composition comprising the virus(es) comprises a sucrose gradient pool of purified virus.
A15. The method of any one of aspects A1-A14, wherein the volume of the liquid composition comprising virus and the chemical viral inactivating agent in the container is within 10%, 5%, 2%, or 1% of the volume calculated to provide the minimum gas-liquid interface size for the container.
A16. The method of any one of aspects A1-A14, wherein the volume of the liquid composition comprising the virus(es) and the chemical viral inactivating agent in the container is within 10%, 5%, 2%, or 1% of the maximum volume recommended by the manufacturer of the container.
A17. The method of any one of aspects A1-A16, wherein an interior surface of the container does not comprise linear low density polyethylene (LLDPE).
A18. The method of any one of aspects A1-A17, wherein the mixing under conditions that produce minimal mechanical stress results in a recovery of virus that is at least 20% more than the recovery of virus under standard mixing conditions.
B1. An inactivated virus preparation produced by the method of any one of aspects A1-A18.
B2. The inactivated virus preparation of aspect B1 for use in treating or preventing a viral infection.

### BRIEF DESCRIPTION OF THE DRAWINGS

The accompanying drawings are not intended to be drawn to scale. The Figures are illustrative only and are not required for enablement of the disclosure. For purposes of clarity, not every component may be labeled in every drawing.

FIG. 1: White particles observed in NIV from routine production derived from precipitated virus during inactivation.

FIGS. 2A-2B: Comparison of stabilization buffers; FIG. 2A: storage at 2-8° C.; FIG. 2B: virus recovery after one freeze-thaw cycle at <−70° C.

FIG. 3: White precipitate formed in constantly agitated bag #2 at 4 h and 48 h compared to non-agitated bag #1. SE-HPLC overlay of samples taken after 4 h and 48 h show the amount of virus loss due to agitation.

FIG. 4: Overlay of SE-HPLC chromatograms during first 48 h of inactivation for bag #1 (left) and bag #2 (right).

FIG. 5: Comparison of SE-HPLC virus recovery over 10 days of inactivation.

FIG. 6: SDS-PAGE analyzes of washed precipitate from bag #2 using silver stain. Additional bands seen in precipitate are multimers derived from the formaldehyde cross linking reaction. All virus specific proteins (M, C and E) are clearly present and no additional bands are observed.

FIG. 7: Inactivated JEV ELISA of NIV samples from both bags.

FIGS. 8A-8B: Influence of mixing speed on virus recovery in Flexboy® bags. FIG. 8A, 0-10 days; FIG. 8B, 0-24 hours.

FIG. 9: Correlation of mixing intensity and virus recovery after 24 h and 48 h inactivation duration.

FIGS. 10A-10D: Comparison of SE-HPLC virus recovery. FIG. 10A, virus recovery in Bag #1, Bag #2. FIG. 10B, virus recovery in Bag #3, Bag #4. FIG. 10C, specific antigen content after neutralization in Bag #1, Bag #2. FIG. 10D, specific antigen content after neutralization in Bag #3, Bag #4.

FIG. 11: Overlays of SE-HPLC chromatograms for bags #1 to 4; Bag #1 and #2: formaldehyde addition in PC bottle; Bag #3 and #4: formaldehyde addition directly into bag.

FIG. 12: Comparison of Flexsafe® bags after neutralization; Bag#1 was mixed at 30 rpm and showed clear precipitate whereas bag #2 was mixed at 6 rpm and did not show any sign of precipitation.

FIG. 13: Virus recovery monitored by SE-HPLC analysis.

FIGS. 14A-14B: Comparison of virus recovery in Flexsafe® and Flexboy® bags after mild (FIG. 14A) and harsh (FIG. 14B) mixing conditions.

FIG. 15: Relative difference between Flexboy® and Flexsafe® bags in % for 6 rpm and 30 rpm mixing speed over time.

FIG. 16: Total virus peak area lost during inactivation in Flexboy® and Flexsafe® bags after mixing with 6 rpm or 30 rpm.

FIG. 17: Pictures of bags incubated at 37° C. for 4 hours show strong precipitation in both cases.

FIGS. 18A-18B: Overlay of SE-HPLC chromatograms of samples after 24 h incubation in bags at 22° C. or 37° C.; FIG. 18A: no mixing; FIG. 18B: constant mixing at 20 rpm on see-saw rocker.

FIG. 19A: Virus recovery by SE-HPLC of bags incubated at 37° C. during the first 24 h without mixing and 20 rpm constant mixing. FIG. 19B: antigen content in NIV determined by inactivated JEV ELISA of a bag incubated at 37° C. in comparison to a control incubated at 22° C.; Note: 37° C. bag with constant mixing was tested negative in ELISA.

FIG. 20: Virus recovery analyzed by SE-HPLC of diluted SGP stirred for a total of 120 min. Mild mixing did not result in virus loss. Harsh mixing did result in ˜14% virus loss. PC-0: mixed by swirling; PC-100: mixed at 100 rpm using magnetic stirrer; PC-300: mixed at 300 rpm.

FIGS. 21A-21C: Virus recovery analyzed by SE-HPLC. FIG. 21A: Virus recovery during inactivation only; FIG. 21B: Overall recovery after initial mixing and inactivation;

FIG. 21C: Antigen content of NIV samples determined by inactivated JEV ELISA.

FIGS. 22A-22C: Correlation analysis of recovered JEV in NIV vs. fill height.

FIG. 23: Correlation of JEV yield vs. liquid fill height and Re_{mod} during virus inactivation in 20 L bag.

FIGS. 24A-24B: Chikungunya virus inactivation (48 h kinetic). FIG. 24A: Impact of constant agitation on CHIKV virus recovery during inactivation. The virus peak decreased by more than 60% for the 30 rpm mixing and only 30% for the 6 rpm mixed sample. FIG. 24B: Overlays of the SE-HPLC results for the three bags after 6 h of inactivation and the starting material.

FIG. 25: TCID50 analysis of samples taken during the first 48 h showed a fast inactivation of Chikungunya virus by formaldehyde with a 99% reduction after ˜9 h and a 99.9% reduction after ˜15 h. Virus titer was below the limit of quantification within after 30 h and complete inactivation was achieved after ˜41 h based on regression analysis.

### DETAILED DESCRIPTION

Disclosed herein are processes for the inactivation of viruses, and compositions comprising such inactivated viruses.

For JEV inactivation, formaldehyde is added to diluted sucrose gradient pool at a concentration of 200 ppm (=0.2 g/L or 6.67 mM). The inactivation process by formaldehyde is time dependent and is completed within 48 h (no active JEV detected by plaque assay). For safety reasons a 0.2 μm filtration step is conducted after 48 h to remove larger particles/aggregates that could potentially contain still infectious particles. Inactivation is continued for additional 8 days in accordance with current guidelines resulting in a total inactivation time of 10 days. The reaction is stopped by the addition of 2 mM sodium metabisulfite (equals 4 mM sulfite), which reacts with the remaining free formaldehyde. Because sulfite reacts with formaldehyde in a 1:1 ratio, the amount of sulfite added cannot completely neutralize the formaldehyde. Consequently, neutralized inactivated virus solution (NIV) still contains up to 50 ppm (=0.05 g/L or 1.66 mM) free formaldehyde.

In the standard inactivation processes, the inactivation solution is placed on a wave mixer and constantly agitated at low rpm (first 10 min: below 10 L volume: 20 rpm at 10° angle; above 10 L: 40 rpm at 12° angle; then for all volumes constant 8 rpm at 8° angle for 240 h). Significant losses were observed throughout the inactivation process, yielding approximately only 34% inactive virus particle recovery for JEV. Moreover, during this inactivation procedure a precipitate was observed, which consists of virus particles.

Further investigational work was done to explore in more detail the root cause of these high losses. During this experimental work it was noted that viruses are very sensitive to mechanical stress mainly caused by the continuous mixing on the rocker over 10 days. A clear relationship between rocking speed and particle recovery was determined.

Based on these data, an alternative mixing strategy was developed to ensure complete mixing of formaldehyde in the inactivation bag with minimal mechanical stress. This novel procedure is mainly based on period manual inversion of the bag for smaller bags or short-term rocking intervals of a defined time for larger bag sizes in which manual inversion is difficult. Surprisingly, the novel inactivation processes resulted a much higher virus particle recoveries after inactivation (>91% recovery for some viruses) and significant yield increases (>4×) compared to standard inactivation procedures. The novel inactivation processes did not show any difference in the kinetics of viral inactivation compared to the standard process.

The methods of inactivating viruses disclosed herein include contacting a liquid composition comprising the virus(es) with a chemical viral inactivating agent in a container, mixing the chemical viral inactivating agent and the liquid composition comprising the virus(es) under conditions of laminar flow but not turbulent flow, and incubating the chemical viral inactivating agent and the liquid composition comprising the virus(es) for a time sufficient to inactivate the virus(es).

The composition comprising virus(es) optionally is a liquid composition. The composition comprising the virus(es) can be the end product of a virus purification process, such as a pool of sucrose gradient fractions of purified virus, also referred to herein as a sucrose gradient pool. Other composition comprising viruses include filtrates, eluates, and other end products of virus purification processes, some of which are described elsewhere herein.

The term “chemical viral inactivating agent” as used herein is any compound that can abolish infectivity of the virus during treatment so that the virus loses its capacity to reproduce without destruction of antigenic and immunogenicity properties. Chemical viral inactivating agents that can be used in the disclosed methods include formaldehyde; enzyme(s); β-propiolactone; ethanol; trifluroacetic acid; acetonitrile; bleach; urea; guanidine hydrochloride; tri-n-butyl phosphate; ethylene-imine or a derivative thereof; an organic solvent, optionally polysorbates such as TWEEN® 20 or TWEEN® 80, TRITON® detergents such as TRITON® X-100, sodium deoxycholate, low pH treatment or sulfobetaine; or a combination thereof.

Containers useful in the disclosed processes can include any commonly used in viral production, e.g., for vaccine production. In some embodiments, the container is a flexible single use bioprocess or bioreactor bag (also referred to herein as a “wave bag”) such as those used in rocking motion bioreactors, which can be obtained, for example, from Sartorius (FLEXSAFE® RM Bags or FLEXBOY® bags) or GE Healthcare Life Sciences (WAVE Cellbag).

The interior film surface of the container to be in direct contact with the virus can have an effect on virus recovery or yield from the viral inactivation process. Preferably, the interior surface of the container is made of the chemical substance with low adsorption capacity and chemically inert, i.e. providing no side effect on the virus. In some embodiments, the interior surface of the container comprises linear low density polyethylene (LLDPE). Alternatively, the interior surface of the container comprises ethylenvinylacetate (EVA). In some embodiments, the container with the interior surface made of ethylenvinylacetate (EVA) is preferred.

The mixing of the chemical viral inactivating agent and the liquid composition comprising the virus(es) is done under conditions that produce minimal mechanical stress, such as conditions of laminar flow, but not turbulent flow. Reducing or avoiding turbulent flow and limiting mechanical stress to the virus(es) in the mixture is demonstrated herein to increase recovery and yield of inactivated virus. When mixing under conditions that produce minimal mechanical stress, such as laminar flow, or reduced or absent turbulent flow, results in a recovery of virus that is at least 20% higher than the recovery of virus under standard mixing conditions. Standard mixing conditions are conditions used in viral inactivation processes without regard to the amount of mechanical stress produced, i.e., art-standard vigorous mixing protocols.

In some embodiments, the recovery of virus is at least 30%, 40%, 50%, 60%, 70%, 80%, 90%, 100%, 110%, 110%, 110%, 110%, 110%, 110%, 110%, 110%, 110%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, or more, greater than the recovery of virus under standard mixing conditions or standard inactivation procedures. In some embodiments, the yield of virus (also referred to as fold increase in yield) is at least 1.5×, 2×, 2.5×, 3×, 3.5×, 4×, 4.5×, 5×, 5.5×, 6×, 6.5×, 7×, 7.5×, or more, greater than the recovery of virus under standard mixing conditions or standard inactivation procedures. In some embodiments, the recovery of virus is at least 40%, 45%, 50%, 55%, 60%, 65%, 70%, 75%, 80%, 85%, 90%, 95%, or even approaching 100% of the viral particles input into the viral inactivation process, e.g., the purified viral particles exposed to the chemical inactivating agent.

A modified Reynolds number (Re_{mod}) describes fluid flow in wave bioreactors (see Eibl & Eibl, 2006 and Eibl et al., 2009). Re_{mod} is a dimensionless number that describes the ratio of internal force to internal friction, and is calculated using the following formula:

$$R {e}_{mo d} = \frac{V * k * C * D}{15 * v * ( 2 * h + B )}$$

wherein V is the volume of the container (e.g., wave bag), k is the mixing rate of the container (e.g., the rocking rate of a rocker on which a wave bag is placed), C is a correlation factor determined for each container based on rocking rate, rocking angle and culture volume, D is a correction factor, which depends on the bag type, v is the kinematic viscosity of the liquid in the container, h is the height of liquid in the container, and B is the width of the container.

Coefficients C and D are correction factors listed respectively in Table 3 and Table 4 of the reference of Eibl & Eibl (2006, p. 212) incorporated herein by its entirety. Table 3 and 4 of Eibl & Eibl (2006) are as follows:

TABLE 3
Correction factor (C) for Wave Bag 20 L

| Rocking | Working volume [L] | | | | |
|---|---|---|---|---|---|
| angle [°] | 2 | 4 | 6 | 8 | 10 |
| 2 | 0.5354 | 0.2892 | 0.2025 | 0.1602 | 0.1323 |
| 4 | 0.819 | 0.5612 | 0.4083 | 0.3138 | 0.2583 |
| 6 | 0.9882 | 0.7628 | 0.5797 | 0.4554 | 0.3747 |
| 8 | 1.000 | 0.894 | 0.7167 | 0.585 | 0.4815 |
| 10 | 1.000 | 0.9548 | 0.8193 | 0.7026 | 0.5787 |

TABLE 4
Correction factor (D) for Wave Bag

| Wave Bag | Correction factor (D) |
|---|---|
| Wave Bag 2 L | 0.0565 |
| Wave Bag 10 L | 0.0398 |
| Wave Bag 20 L | 0.312 |
| Wave Bag 100 L | 0.015 |
| Wave Bag 200 L | 0.0489 |

Kinematic viscosity (v) is the ratio of absolute (or dynamic) viscosity to density, and can be calculated using the following formula:

v=μ/ρ

wherein v is kinematic viscosity (m²/s), μ is absolute or dynamic viscosity (N s/m²), and ρ is density (kg/m³).

Dynamic viscosity (μ) is measured as the resistance to flow when an external and controlled force (pump, pressurized air, etc.) forces oil through a capillary (ASTM D4624), or a body is forced through the fluid by an external and controlled force such as a spindle driven by a motor. In either case, the resistance to flow (or shear) as a function of the input force is measured, which reflects the internal resistance of the sample to the applied force, or its dynamic viscosity. There are several types and embodiments of absolute viscometers, the Brookfield rotary method is the most common.

Density (ρ) can be measured by a laboratory balance and high precision pipettes.

Reducing or avoiding turbulent flow and maximizing laminar flow can be achieved by performing the mixing under conditions that result in a modified Reynolds Number (Re_{mod}) of less than 1000, less than 950, less than 900, less than 850, less than 800, less than 750, less than 700, less than 650, less than 600, less than 550, less than 500, less than 450, less than 400, less than 350, less than 300, less than 250, or less than 200. Optimal conditions are to perform the mixing under conditions that result in a modified Reynolds Number (Re_{mod}) of less than 1000 but different container geometries, fill conditions, mixing rates and angle, and so on can influence the Re_{mod} at which the laminar-to-turbulent flow occurs. See Eibl & Eibl, 2006 and Eibl, et al., 2009.

In some embodiments, minimizing the gas-liquid interface helps to increase virus yield or recovery. Thus, in some embodiments the volume of the composition comprising viruses and the chemical viral inactivating agent in the container is within 20%, 19%, 18%, 17%, 16%, 15%, 14%, 13%, 12%, 11%, 10%, 9%, 8%, 7%, 6%, 5%, 4%, 5%, 2%, or 1% of the volume calculated to provide the minimum gas-liquid interface size for the container. In other embodiments, the volume of the composition comprising viruses and the chemical viral inactivating agent in the container is within 20%, 19%, 18%, 17%, 16%, 15%, 14%, 13%, 12%, 11%, 10%, 9%, 8%, 7%, 6%, 5%, 4%, 5%, 2%, or 1% of the maximum volume recommended by the manufacturer of the container.

Thus to maximize laminar flow and minimize turbulent flow in accordance with the disclosed methods, mixing can be accomplished by inverting the container a limited number of times during the incubation of the chemical viral inactivating agent and the liquid composition comprising viruses. In some embodiments, the container is inverted not more than 1, 2, 3, 4 or 5 times during the period of incubation.

Alternatively, the mixing can be accomplished by subjecting the container to rocking, rotation, orbital shaking, or oscillation for not more than 5 seconds, 10 seconds, 15 seconds, 30 seconds, 45 seconds, 1 minute, 2 minutes, 3 minutes, 4 minutes, 5 minutes, 6 minutes, 7 minutes, 8 minutes, 9 minutes, or 10 minutes at not more than 2 rpm, 3 rpm, 4 rpm, 5 rpm, 6 rpm, 7 rpm, 8 rpm, 9 rpm, or 10 rpm, during the period of incubation.

Another way to increase recovery and/or yield of virus is to limit the amount of mixing during the inactivation method. For example, in some embodiments the mixing is performed only within the first 10 minutes, 20 minutes, 30 minutes, 40 minutes, 50 minutes, or 60 minutes after the contacting of the viruses and the agent in the container. In other embodiments, no mixing is performed after 2 hours, 4 hours, 8 hours, 12 hours, 24 hours, or 48 hours from the contacting of the viruses and the agent in the container.

While the disclosed viral inactivation methods favorably increase recovery and/or yield of virus, they do not require substantial additional time. In some embodiments, the inactivation of the viruses is completed in a time period that is not more than 1%, 2%, 3%, 4%, 5%, 6%, 7%, 8%, 9%, or 10% longer than the time period for inactivation of the same viruses using the same chemical viral inactivation agent without any restriction on mixing (e.g., standard mixing conditions).

In the disclosed viral inactivation methods, the chemical viral inactivating agent and the liquid composition comprising viruses are incubated for 1, 2, 3, 4, 5, 6, 7, 8, 9, 10, 11, 12, 13, 14, 15, 16, 17, 18, 19, or 20 days. After any such time of incubation, a sample of the mixture can be withdrawn from the container to analyze the completeness of viral inactivation, yield, and/or recovery.

Typically, the chemical viral inactivating agent and the composition comprising viruses are incubated at about 10° C. to about 30° C., e.g., at 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C. It also is possible to start the viral inactivation process at one temperature, and shift temperature one or more times during the viral inactivation process.

Also provided are methods for purification and inactivation of viruses. Such methods include purifying a population of viruses as described herein, followed by inactivating the purified viruses by contacting a liquid composition comprising purified viruses with a chemical viral inactivating agent in a container, mixing the chemical viral inactivating agent and the liquid composition comprising viruses under conditions of laminar flow but not turbulent flow, and incubating the chemical viral inactivating agent and the liquid composition comprising viruses for a time sufficient to inactivate the viruses.

Aspects of the disclosure relate to processes for inactivating a virus. Any virus for which an inactivated virus preparation is desired may be compatible with aspects of the disclosure. The terms “virus,” “virus particle,” “viral particle,” and “virion” may be used interchangeably and refer to a virus comprising genetic material surrounded by a protein coat (capsid), and optionally a lipid envelope. In general, viruses may be classified based on the virus genetic material contained within the protein coat and the method by which the virus is able to generate message RNA (mRNA) in an infected cell (a host cell).

For example, the virus may be a DNA virus or an RNA virus. In some embodiments, the virus is a retrovirus meaning the virus reverse transcribes its nucleic acid through an intermediate during replication. In some embodiments, the virus is a double stranded DNA (dsDNA) virus, a single stranded DNA (ssDNA) virus, a double stranded RNA (dsRNA) virus, a positive strand single stranded RNA (+ssRNA) virus, a negative strand single stranded RNA (−ssRNA) virus, a single stranded RNA retrovirus (ssRNA-RT), or a double stranded DNA retrovirus (dsDNA-RT).

A virus may also be classified based on the type of host cell that it is capable of infecting. As used herein, a virus is capable of infecting a cell if it is able to enter the cell, replicate and be released from the cell. In some embodiments, the virus is capable of infecting eukaryotic cells. In some embodiments, the virus is an animal virus (i.e., capable of infecting animal cells). In other embodiments, the virus is a plant virus (i.e., capable of infecting plant cells).

The processes described herein may be used to inactivate live viruses. In some embodiments, the virus is an attenuated live virus. For example, the virus may have reduced infectivity, virulence, and/or replication in a host, as compared to a wild-type virus. In some embodiments, the virus is a mutated or modified virus, for example the nucleic acid of the virus may contain at least one mutation relative to the wild-type virus. In some embodiments, the virus is a recombinant live virus, meaning a virus that is generated recombinantly and may contain nucleic acid from different sources.

In some embodiments, the virus belongs to one of the following families: Flaviviridae, Togaviridae, Paramyxoviridae, Orthomyxoviridae, Filoviridae, Arenaviridae, Rhabdoviridae, or Coronaviridae. Particularly preferred viruses to be used with the processes described herein include Japanese encephalitis virus, Zika virus, Yellow Fever virus, Dengue virus, Chikungunya virus and Measles virus.

Aspects of the invention described herein relate to aseptic processes. As used herein, the term “aseptic” refer to compositions, processes, and conditions that are free from any contaminating living organisms. In some embodiments, each step of the process is performed under aseptic conditions such that the resulting virus preparation may be free from other organisms.

The processes described herein involve, in some aspects, providing a liquid medium comprising a plurality of viruses for inactivation. Viruses may be produced or provided by any method known in the art. For example, the virus may be produced by propagating in a live host, an embryonic egg, tissue culture or cell line, such as in the EB66® cell line. Selection of the method for producing the virus will depend on various factors such as the virus and type of host cell it is capable of replicating and the amount of virus production desired.

In certain embodiments, the virus is propagated in cell or tissue culture. Any cell that is permissive (capable of being infected with the virus) for entry and replication of the virus can be used for virus propagation. In some embodiments, the cells are primary cells (e.g., cells that have been isolated from a host organism). In some embodiments, the cells are from a cell line. In some embodiments, the cell line is derived from cells of a mammal (such as a human or non-human mammal), a bird, an insect, or a plant. In some embodiments, the cells of the cell line are MDCK cells, CAP cells, AGE1.CR, EB66® cells, MRC-5 cells, Vero cells, Vero-Hisα cells, HeLa cells, HeLa-S3 cells, 293 cells, PC12 cells, CHO cells, 3T3 cells, PerC6 cells, chicken embryonic fibroblasts (CEFs), PBS-1 cells, QOR/2E11vcells, SogE cells, MFF-8C1 cells, or diploid avian cells. In some embodiments, the cells of the cell line are cells that grow in suspension and do not adhere. In some embodiments, the diploid avian cells are derived from avian stem cells. In some embodiments, the diploid avian cells are duck cells. In some embodiments, the cells are of the EB66® cell line.

Following viral replication in a cell or cell population, the virus may be released into a liquid medium surrounding the infected cell. In some embodiments, the host cell may be lysed (e.g., enzymatically, mechanically) to release the virus into the liquid medium. The type of liquid medium into which the virus is released will depend on the type of host cell and viral propagation method used. In some embodiments, the liquid medium contains serum, plasma, blood, extracellular fluid, allantoic fluid, amniotic fluid, yolk sac, buffer, or cell or tissue culture medium. Any cell or tissue culture medium that supports growth of the cell or cell population may be used.

In some embodiments, the cells are grown as a monolayer on a culture substrate, such as a flask, dish or plate. In such embodiments, the virus is harvested from the cells by removing the culture medium from the cells. In some embodiments, the cells are lysed to release the virus into the culture medium and the culture medium is collected to harvest the virus. In other embodiments, the cells are grown in suspension in which the cells are floating or only lightly adherent to the culture substrate. In some embodiments, the culture substrate may be a rolling flask, shaker flask, spinner flask, or bioreactor. In yet other embodiments, the cells are grown in a mixed culture in which a portion of the cells are adherent to the culture substrate and a portion of the cells are floating and non-adherent. In some embodiments, the cells and the virus are both present in the liquid medium.

Methods for culturing cells will be evident to one of skill in the art. See, e.g., Harrison & Rae. General Techniques of Cell Culture, Cambridge University Press, Cambridge, United Kingdom, 1997.

In some embodiments, the liquid medium containing the virus is subjected to one or more pre-purification steps. In some embodiments, one or more pre-purification steps may be used, for example, to reduce the presence of one or more impurities or contaminants, remove host cells or fragments thereof, enhance virus yield, and/or reduce total processing time.

In some embodiments, any host cells or fragments thereof may be separated or removed from the liquid medium comprising the virus by any suitable means known in the art. In some embodiments, host cells are removed by centrifugation or filtration of the liquid medium. Centrifugation may be performed at a speed and duration that results in separation of host cells or fragments thereof from the virus. For example, the host cells or fragments thereof form a pellet while the virus remains in the liquid medium. Alternatively or in addition, filtration methods, such as membrane filtration, may be used to remove host cells or fragments thereof from the liquid medium containing the virus (e.g., ultrafiltration). In some embodiments, a filter membrane is selected such that the virus is able to pass through the filter but host cells and fragments thereof remain trapped in the membrane.

In some embodiments, the one or more pre-purification steps involve degrading host cell genomic DNA in the liquid medium comprising the virus. In some embodiments, the host cell genomic DNA is degraded by enzymatic treatment. Any DNA degrading enzyme may be compatible with the processes described herein. In some embodiments, the enzyme is a nuclease. In some embodiments, the nuclease degrades both DNA and RNA. Non-limiting examples of nucleases include, without limitation, BENZONASE®, DNAse I, DNAse II, Exonuclease II, micrococcal nuclease, nuclease P1, nuclease S1, phosphodiesterase I, phosphodiesterase II, RNAse A, RNAse H, RNAse T1, or T7 endonuclease. In some embodiments, the DNA degrading enzyme treatment reduces or eliminates the presence of DNA fragments larger than about 200 base pairs in length. The enzyme concentration, incubation time, and temperature to degrade nucleic acid in the liquid medium comprising the virus will be evident to one of skill in the art. In some embodiments, the ion concentration (e.g. Mg2+, Mn2+) and/or pH of the liquid medium comprising the virus may also be optimized to enhance or reduce activity of the enzyme. DNA degrading enzymes may be isolated or obtained from any source known in the art, for example the enzyme may be a microbial, plant, or mammalian enzyme; recombinantly produced; and/or commercially available.

In some embodiments, the one or more pre-purification steps involve ultrafiltration and/or diafiltration of the liquid medium comprising the virus. As used herein, “ultrafiltration” refers to a method of separating components of a mixture based on the size or molecular weight of the components by passing the liquid medium through a semi-permeable membrane. Components that have a larger molecular weight than the pore size (the molecular weight cutoff (MWCO)) of the semi-permeable membrane are retained on the membrane, while components of smaller molecular weight are allowed to pass through the membrane. As used herein, “diafiltration” refers to a method of reducing the concentration of a component, such as an impurity or contaminant, in a mixture, and/or exchanging buffers.

Diafiltration may be performed by any of a number of methods, for example, continuous diafiltration, discontinuous diafiltration, or sequential diafiltration. In some embodiments, ultrafiltration and diafiltration methods are performed concurrently or sequentially.

In some embodiments, the ultrafiltration and diafiltration are performed using tangential flow filtration. As used herein, “tangential flow filtration,” also referred to as “cross flow filtration,” is a filtration method in which the feed stream (i.e., the liquid medium containing the virus) is tangential to the filter membrane. In some embodiments, the tangential flow filtration is performed using a hollow fiber membrane. The feed stream is fed into the tubular fiber and components of the feed that are smaller than the MWCO of the membrane are allowed to pass through and out of the stream, whereas larger components are maintained in the stream and may be recirculated through the system. Additional liquid medium or an alternative buffer may be continuously added to the stream at the same rate as removal of small components of the mixture, thereby maintaining a consistent concentration of the virus. In some embodiments, the liquid medium comprising the virus is subjected to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or at least 30 volume exchanges of liquid medium or an alternative buffer. Non-limiting examples of alternative buffers include phosphate buffered solution (PBS), Dulbecco's phosphate-buffered saline (DPBS), Earle's balanced salt solution (EBSS), Hank's balanced salt solution (HBSS), or water.

In some embodiments, the MWCO of the membrane is at least 500 kilodaltons (kDa), 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, or at least 900 kDa. In some embodiments, the MWCO of the membrane is greater than or equal to 750 kDa.

In some aspects, providing a liquid composition comprising viruses can include layering the liquid medium comprising the virus on top of a sucrose density gradient and centrifuging it to produce a zone of virus separated from zones of impurity. Fractions of the sucrose gradient can then be taken, with those fractions containing virus used as is or pooled to form a sucrose gradient pool.

For example, as described in WO2017/109223 and WO2017/109224, purification of infectious virus particles can includes the steps of providing a crude harvest (a) comprising virus particles and impurities, wherein the impurities are generated from growing said virus particles on a cell substrate; reducing impurities from the crude harvest (a) by precipitation with an agent comprising a protamine salt, preferably a protamine sulphate, to obtain a virus preparation (b); and further purifying the virus preparation (b) by an optimized sucrose density gradient centrifugation to obtain a virus preparation (c) comprising the infectious virus particles. The crude harvest (a) is subjected to one or more pre-purification step(s) prior to the precipitation step. In some embodiments, the one or more pre-purification step(s) comprises digesting host cell genomic DNA in the crude harvest (a) comprising the virus particles and impurities by enzymatic treatment. In some embodiments, the one or more pre-purification step(s) comprises filtration, ultrafiltration, concentration, buffer exchange and/or diafiltration.

As described in WO2017/109223 and WO2017/109224, adding protamine sulfate to a virus harvest produced on a cell substrate removed not only contaminating DNA derived from host cells, but surprisingly also virtually eliminated immature and otherwise non-infectious virus particles from the preparation.

The concentration of protamine sulphate used is about 1 to 10 mg/ml, preferably about 1 to 5 mg/ml, more preferably about 1 to 2 mg/ml, more preferably 1.2 to 1.8 mg/ml, more preferably 1.4 to 1.6 mg/ml. Specific protamine sulfate molecules useful in the methods disclosed herein are include SEQ ID NO:1 of WO2017/109224, and the molecules recited in the third paragraph on page 12 of WO2017/109224.

The process may also include the use of a sucrose gradient, preferably an optimized sucrose gradient. The sucrose gradient is preferably optimized for the removal of protamine sulfate, also for the removal of immature viral particles or other viral particles which are non-infectious or host cell proteins or nucleic acids (DNA, RNA, mRNA, etc.) or other host cell debris. The optimized sucrose gradient includes at least two, at least three, at least four layers of sucrose solutions with different densities. In one embodiment, the virus preparation to be purified is provided in a sucrose solution which has a density of about 8%, about 9%, about 10%, about 11%, about 12% sucrose (w/w), preferably about 10%. In one embodiment, one sucrose solution in the gradient has a density of about 45%, about 46%, about 47%, about 48%, about 49%, about 50%, about 51%, about 52%, about 53%, about 54%, about 55% sucrose (w/w), preferably about 50%. In one embodiment, one sucrose solution in the gradient has a density of about 30%, about 31%, about 32%, about 33%, about 34%, about 35%, about 36%, about 37%, about 38%, about 39%, about 40% sucrose (w/w), preferably about 35%. In one embodiment, one sucrose solution in the gradient has a density of about 10%, about 11%, about 12%, about 13%, about 14%, about 15%, about 16%, about 17%, about 18%, about 19%, about 20% sucrose (w/w), preferably about 15% sucrose. In a preferred embodiment, the sucrose gradient comprises three layers of sucrose solutions of about 50%, about 35% and about 15% (w/w) sucrose and the virus composition to be purified is contained in about 10% (w/w) sucrose.

In some aspects, providing a liquid composition comprising viruses can include contacting the liquid medium comprising the virus with a solid-phase matrix. In some embodiments, the liquid medium comprising the virus is contacted with a solid-phase matrix by batch adsorption. As used herein, “batch adsorption” refers to a method in which a solid-phase matrix is added to a liquid phase mixture of components (e.g., the liquid medium comprising the virus) including a molecule for which purification is desired (e.g., a virus). In some embodiments, the solid-phase matrix is suspended in a buffer solution referred to as a slurry. The solid-phase matrix adsorbs components of the mixture. Subsequently, the solid-phase matrix and the adsorbed components may be separated from the mixture using any method known in the art, such as centrifugation, filtration, or flocculation. In some embodiments, the molecule for which purification is desired (e.g., a virus) is adsorbed to the solid-phase matrix. In other embodiments, impurities or contaminants are adsorbed to the solid-phase matrix and the molecule for which purification is desired remains in the liquid phase. General batch adsorption methods and considerations can be found, for example, in Scopes R. K. Protein Purification: Principles and Practice, 3^{rd} Edition, 1994, Springer Advanced Texts in Chemistry, New York, N.Y.

In some embodiments, the solid-phase matrix comprises a matrix and a ligand that binds components of a mixture. In some embodiments, the matrix is SEPHAROSE® or agarose, such as highly cross-linked agarose. In some embodiments, the solid-phase matrix comprises a ligand-activated core containing the ligand that binds components of a mixture and an inactive shell. In some embodiments, the inactive shell surrounds the matrix and the core ligand and comprises pores with a MWCO. In general, the pores of the inactive shell prevent binding of the virus with the ligand of the solid-phase matrix and allow entry of components of size less than the MWCO to enter the inactive shell and interact with the ligand. In some embodiments, the MWCO of the inactive shell is at least 500 kilodaltons (kDa), 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, or at least 900 kDa. In some embodiments, the MWCO of the inactive shell is greater than or equal to 700 kDa. In some embodiments, the pores of the inactive shell allow entry of impurities into the ligand-activated core of the solid-phase matrix. In some embodiments, impurities interact with or bind to the ligand-activated core. In some embodiments, the impurities may interact with or bind to the ligand-activated core by any type of interaction known in the art. In some embodiments, the impurities may interact with or bind to the ligand-activated core by cation, anion, hydrophobic, or mixed interactions.

In some embodiments, the ligand of the solid-phase matrix is octylamine, diethylaminoethyl, quarternary ammonium, or sulfonate. Non-limiting examples of solid-phase matrices that may be compatible with the processes described herein include, without limitation, CAPTO® Core 700, CAPTO® DEAE, CAPTO® MMC, CAPTO® Q, CAPTO® S, FRACTOGEL® TMAE, Hyx T II, Q SEPHAROSE® Fast Flow. In some embodiments, the solid-phase matrix is CAPTO® Core 700.

In some embodiments, the solid-phase matrix is suspended in a buffer solution as a slurry prior to combining with the liquid medium comprising the virus. In some embodiments, the solid-phase matrix is combined with the liquid medium comprising the virus as a slurry at a final concentration between 2.5% (v/v)-30% (v/v), 5% (v/v)-20% (v/v), or 7.5% (v/v)-15% (v/v). In some embodiments, the slurry is added at a final concentration of approximately 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 10.5%, 11%, 12%, 13%, 14%, 15%, 16%, 17%, 18%, 19%, 20%, 21%, 22%, 23%, 24%, 25%, 26%, 27%, 28%, 28%, 29%, or 30% (v/v). In some embodiments, the slurry is added at a final concentration of approximately 10% (v/v).

Conditions, including the duration, temperature, and mode of contact between the solid-phase matrix and the liquid medium comprising the virus, may be varied in order to enhance recovery of the virus and enhance binding and removal of impurities from the liquid medium. In some embodiments, the solid-phase matrix is contacted or incubated with the liquid medium comprising the virus at a temperature between 15° C.-30° C., such as 17° C.-27° C., or 20°-25° C. In some embodiments, the solid-phase matrix is contacted or incubated with the liquid medium comprising the virus at room temperature. In some embodiments, the solid-phase matrix is contacted or incubated with the liquid medium comprising the virus at a temperature of 15° C., 16° C., 17° C., 18° C., 19° C., 20° C., 21° C., 22° C., 23° C., 24° C., 25° C., 26° C., 27° C., 28° C., 29° C., or 30° C.

In some embodiments, the solid-phase matrix is contacted or incubated with the liquid medium comprising the virus for a duration between 1 and 5 hours, 1 and 10 hours, 1 and 24 hours, 5 and 10 hours, 10 and 15 hours, or between 15-24 hours. In some embodiments, the solid-phase matrix is contacted or incubated with the liquid medium comprising the virus for approximately 1, 2, 3, 4, 5, 6, 7, 8, 9, 10, 11, 12, 13, 14, 15, 16, 17, 18, 19, 20, 21, 22, 23, or 24 hours. In some embodiments, the solid-phase matrix is contacted or incubated with the liquid medium comprising the virus for approximately 2 hours.

Following batch adsorption, the solid-phase matrix and any bound components may be removed from the liquid phase by any method known in the art, such as centrifugation, filtration, or flocculation. In some embodiments, the solid-phase matrix and any bound components are removed by filtration, such as by any of the filtration methods described herein. In some embodiments, the solid-phase matrix and any bound components are removed by membrane filtration using a membrane with a pore size of at least 0.5, 0.6, 0.7, 0.8, 0.9, 1, 1.1, 1.2, 1.3, 1.4, 1.5, 1.6, 1.7, 1.8, 1.9, or at least 2.0 μm. In some embodiments, the pore size of the membrane is greater than or equal to 1.0 μm. The solid-phase matrices used in the processes described herein may be regenerated (e.g., cleaned and re-sterilized) and used for batch adsorption again.

Virus preparations produced using any of the processes described herein may be further subjected to additional processing steps, including additional filtration steps and/or lyophilization. The virus preparation may also be subjected to analysis for purity of the preparation. For example, the virus preparations may also be assessed for the presence of impurities and contaminants, host cell genomic DNA, and/or host cell proteins. The purity of a virus preparation may be assessed using any method known in the art, such as size exclusion chromatography (SEC), optical density at different wavelengths, protein gel electrophoresis (e.g., SDS-PAGE), Western Blotting, ELISA, PCR, and/or qPCR.

In some embodiments, the virus preparation is assessed for the amount of residual impurities or contaminants. In some embodiments, the amount of residual impurities or contaminants is compared to the amount of impurities or contaminants at an earlier stage in the purification process. In some embodiments, the relative reduction of impurities in the final virus preparation is between 60-95% relative to the presence of impurities at an earlier stage in the purification process. In some embodiments, the relative reduction of impurities in the final virus preparation is approximately 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95%. In some embodiments, the final virus preparation contains less than 5% impurities or contaminants. In some embodiments, the final virus preparation contains less than 5, 4, 3, 2, 1, 0.9, 0.8, 0.7, 0.6, 0.5, 0.4, 0.3, 0.2, or less than 0.1% impurities. In some embodiments, the final virus preparation contains less than 1% impurities.

Any of the processes described herein may be used in the manufacture of a composition comprising inactivated virus for administration to a subject. In some embodiments, the subject is a mammalian subject, such as a human or a non-human animal, including livestock, pets or companion animals. In some embodiments, the composition may be administrated to a subject in need of immunization against the virus or similar virus as that of the virus preparation. In some embodiments, the virus preparations or compositions comprising viruses inactivated using the processes described herein are for treating or preventing infection with the virus or a similar virus as that of the virus preparation.

The virus preparations or compositions of viruses inactivated using the processes described herein may be administered to a subject by any route known in the art. In some embodiments, the preparations or compositions may be administered via conventional routes, such as parenterally. As used herein, “parenteral” administration includes, without limitation, subcutaneous, intracutaneous, intravenous, intramuscular, intraarticular, intrathecal, or by infusion.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Scale-Down Model of Virus Inactivation Process with JEV

In the commercial production of IXIARO® the infectious virus particles are inactivated by incubation of the purified active Japanese Encephalitis virus (also referred herein as JEV) material with formaldehyde for 10 days. Sucrose gradient pools from both runs are pooled and diluted in a 20 L Flexboy® bag. Before and after addition of formaldehyde the diluted SGP is mixed thoroughly on a rocker (20 rpm or 40 rpm depending on volume). Afterwards the material is constantly agitated at 8 rpm during the whole 10 day inactivation period with a 0.2 μm filtration step after 48 h into a fresh 50 L bag. During the formaldehyde neutralization by sodium metabisulfite addition the material is again heavily mixed (33 rpm) for 30-45 minutes. The neutralized inactivated virus (NIV) material is then diluted to drug substance level with PBS and once more 0.2 μm filtered into a fresh 50 L bag.

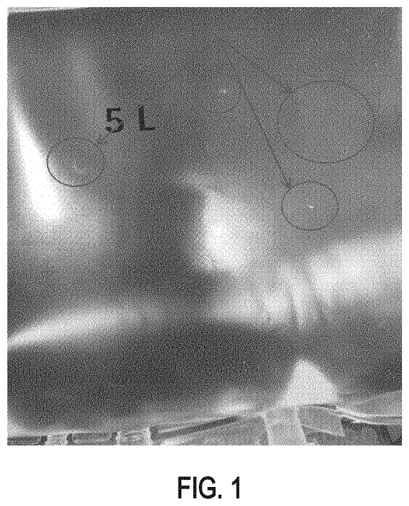

During inactivation a white precipitate is formed that is easy visible by visual inspection (FIG. 1), which is removed by the multiple 0.2 μm filtration steps. Nevertheless these particles will be formed in NIV upon storage probably due to precipitation reactions already started during inactivation (sub-visual particles below 200 nm are not removed by the filtration steps). Because the sucrose gradient pool contains highly purified virus this precipitate is mainly composed of virus particles. Consequently the product recovery during the inactivation step at manufacturing scale is on average only 34±11% step yield. Accordingly a scale-down model of viral inactivation was devised to test various parameters of the inactivation process to identify the source of the low yield of virus.

### General Considerations for the Scale-Down Model

The surface to volume ratio is critical for a correct scale-down model. Sartorius Flexboy® bags are available in discrete sizes (150 mL, 250 mL, 500 mL, 1 L, 3 L). At a maximum filling volume of 80% of nominal volume the surface to volume ratios are given in Table 1.

TABLE 1
Comparison of different Flexboy ® bags

| Bag size nominal | Bag surface | Filling | Surface to volume |
|---|---|---|---|
| (mL) | (cm²) | volume (mL) | ratio (cm² per mL) |
| 150 | 275 | 150 | 1.83 |
| 250 | 329 | 250 | 1.32 |
| 500 | 452 | 500 | 0.90 |
| 1000 | 707 | 1000 | 0.71 |
| 3000 | 1346 | 3000 | 0.45 |
| 20,000 | 4826 | 20,000 | 0.24 |
| 50,000 | 8106 | 50,000 | 0.16 |

The surface to volume ratios become more favorable when using larger bags as the bag surface increases by a factor of 5 whereas the volume increases by a factor of 20. Assuming a constant amount of virus adsorbs per cm² of bag surface the relative losses by unspecific adsorption will thus be much lower when using larger volumes and larger bags. This may be considered when comparing the small-scale results with final production scale.

In JEV routine production the inactivation is done in a 20 L bag with on average 13.1 L starting volume (=65% of loading capacity) resulting in a surface to volume ratio of 0.37 during the first 2 days of inactivation.

When using a 500 mL bag to have the same ratio would require 1221 mL filling volume which cannot be achieved.

It was therefore decided to use a 10× higher surface to volume ratio for the scale down: 130 mL diluted IVS in a 500 mL bag resulting in a surface to volume ratio of 3.48.

### Materials and Equipment

### Materials

Sartorius Flexboy® bag 500 mL (# FFB102670; Lots: T0000944, 15T31612, 10T25068)
Sartorius Flexsafe® bag 500 mL (# FLS 130011; Lot: P0001557)
InVitro Scientific Biotainer PC 125 mL (# IVSP-125-1; Lot: 1162255)
Thermo Square PETG Bottles 250 mL (#2019-0250; Lot: 1188362)
Thermo Square PETG Bottles 1 L (#2019-1000; Lot: 1213015)
Nunc Cryo Tube Vials 1.8 mL (#375418; Lot: 121215)
Thermo Chromacol 300 μL HPLC vials (#03-FISC; Lot: 887119324132419)

### Equipment

Heidolph DuoMax 1030 rocker
Stuart mini see-saw rocker SSM4
Dionex UltiMate 3000 HPLC system with Chromeleon software (Dionex, Austria)
   Solvent Rack SR-3000, without vacuum degasser
   Pump LPG-3400A, analytical low pressure gradient pump
   Autosampler WPS-3000 TSL, analytical autosampler—temperature controlled
   Column compartment TCC-3200, temperature controlled
   PDA-Detector PDA-3000 or VWD-Detector VWD-3400

### Size Exclusion-HPLC

Samples were analyzed using a Superose6 Increase 10/300 SEC column coupled to a Dionex Ultimate 3000 HPLC system.

HPLC Method Parameters:

| | |
|---|---|
| Solvents (mobile | Channel A-1 × PBS, 250 mM NaCl |
| phase): | Channel B-High Purity water, grade 1 (ISO 3696: |
| | 1987) |
| | Channel C-20% ethanol in water |
| Run time: | For samples and standards: 45-50 minutes |
| | (for samples containing no BSA and sucrose |
| | concentrations of <1% w/v in the sample matrix, the |
| | run time can by reduced to 40 min) |
| | For Blank runs and column-cleaning runs: 40-45 min |
| Retention time | approx. 0.3 column volumes (varies per column; |
| JEV: | ca. 8.3 min for the column used) |
| Retention time | main (monomer) peak: approx. 0.7 column volumes |
| BSA: | (varies per column; ca. 16.7 min for the column |
| | used) |
| Flow (isocratic): | 1 mL/min |
| Flow ramp | 0.2 mL/min² |
| Maximal pressure | 50 bar |
| Autosampler | 8° C. |
| temperature: | |
| Injection volume: | 100 μL |
| Sample draw speed: | 5 μL/sec |
| Column | 25° C. |
| compartment: | |
| UV Detector: | PDA or VWD at 214 and 280 nm |
| | Data collection rate minimum 2.5 Hz |

### Analysis of JEV Precipitate Using SDS-PAGE/Silver Stain

SDS-PAGE/silver stain was used for analysis of recovered JEV precipitate during inactivation. In short, JEV precipitate was collected by centrifugation, washed twice with PBS buffer, re-suspended in LDS buffer and stored frozen at −20° C. until analysis. For loading, the samples were thawed, diluted 1: 4 with LDS buffer and heat-denatured at 70° C. for 5 minutes. Samples were separated on 4-12% Bis-Tris Gels (NuPAGE), silver stained using the Invitrogen Silver Express staining kit according the manufacturer's instructions and compared to a JEV sucrose gradient pool reference sample.

### Stabilization of in Process Samples for Analysis

For reliable analysis of in-process samples either by SE-HPLC or by inactivated JEV ELISA stabilization of the samples is required.

Two stabilizing buffers were evaluated for virus recovery during storage at 2-8° C. and up to 2 freeze-thaw cycles at <−70° C.:

Stabilizing buffer A: 5% glycerol, 50 μg/mL BSA in PBS (prepared as 10× stock); Stabilizing buffer B: 5% sucrose, 50 μg/mL BSA in PBS (prepared as 10× stock).

Both buffers were tested using a 24 h inactivation sample drawn from a Flexboy® bag incubated without mixing after an initial 10 min, 10 rpm step.

Virus stability was assessed by comparing SE-HPLC virus recovery of stabilized samples after 1 and 9 days stored at 2-8° C. or up to 2 freeze-thaw cycles at <−70° C.

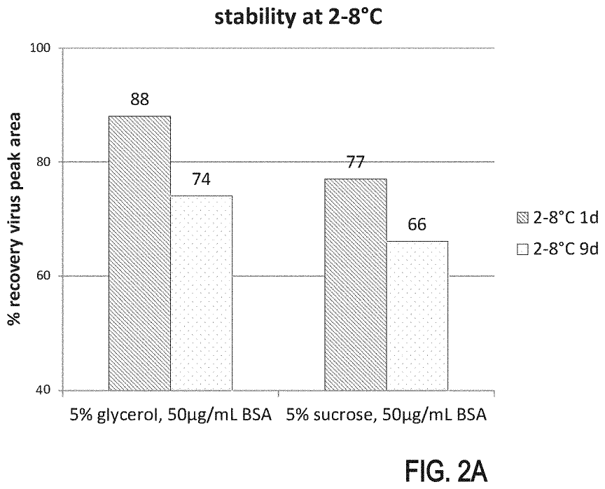

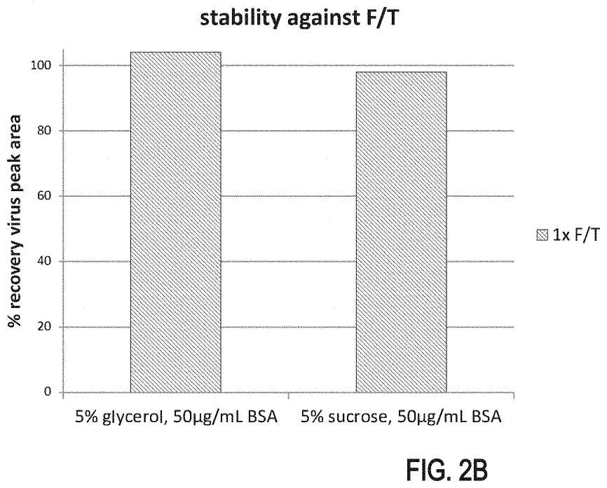

While both buffers showed high stability against freezing (FIG. 2B) the buffer containing glycerol and BSA showed higher stability at 2-8° C. (FIG. 2A).

Therefore buffer A (10× stock: 50% glycerol, 500 μg/mL BSA in PBS buffer) was used in all further experiments for stabilization of samples. Furthermore all sample which were not immediately analyzed were stored frozen at <−70° C. until analysis.

### Example 2: Influence of Constant Rocking on Virus Yield in Flexboy® Bags

Sucrose gradient pool (SGP) from JEV production lot JEV17D54 Bottle B09 was analyzed by SE-HPLC with a virus peak area of 165 mAU*min. The SGP was diluted to 10 mAU*min with PBS buffer in a 250 mL PETG bottle and mixed by stirring for 3 min at 100 rpm. After addition of formaldehyde into the PETG bottle the IVS was mixed at 200 rpm for 3 min and subsequently transferred to two 500 mL Flexboy® bags. A 60 mL air cushion was added to each bag using a syringe and the bags were incubated for 10 days either constantly agitated on a see-saw rocker at 20 rpm, 8° angle, 22° C. without light (Bag #2) or put in a dark box next to the see-saw rocker (Bag #1).

Samples were drawn from each bag using syringes, immediately neutralized with sodium metabisulfite and analysed by SE-HPLC for virus peak recovery. For Bag #1 before each sampling point the bag was inverted gently to assure homogeneity before sampling.

Around 3-4 hours after start a large amount of white precipitate was observed in the constantly agitated Bag #2 but no precipitate could be observed in Bag #1. This macroscopic observation was confirmed by SE-HPLC analysis as the virus recovery for Bag #2 was only 56% compared to Bag #1 after 4 h.

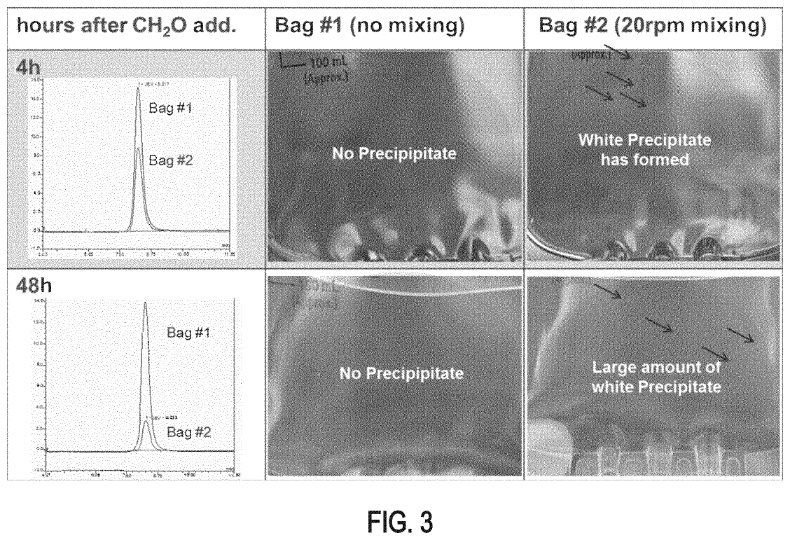

Pictures of both bags taken after 4 h and 48 h of incubation are shown in FIG. 3 together with overlays of the corresponding SE-HPLC chromatograms. After 48 h incubation the relative virus content of bag #2 was only 16% compared to bag #1.

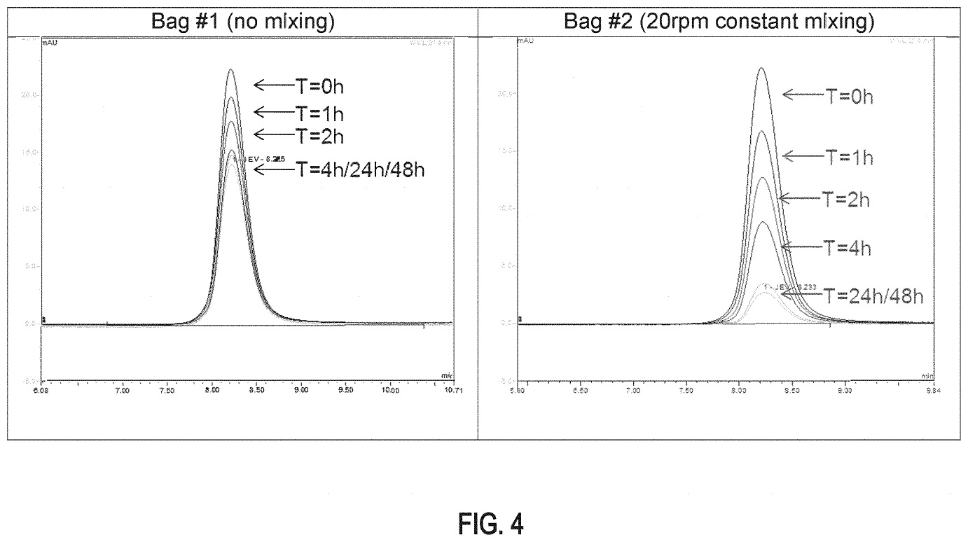

The loss of virus over the first 48 h of incubation can be visualized by plotting overlay SE-HPLC chromatograms for both bags. In FIG. 4 the overlay of chromatograms for bag #1 show an initial phase of decreasing peak area during the first 2-4 h but no more virus loss afterwards and ˜65% recovery compared to the start. On the other hand because of the constant mechanical stress the virus in bag #2 starts to precipitate resulting in significantly higher losses of virus peak area during the first 24 h of inactivation (the time required for the formaldehyde cross-linking to be complete) and only ˜10% recovery compared to the start.

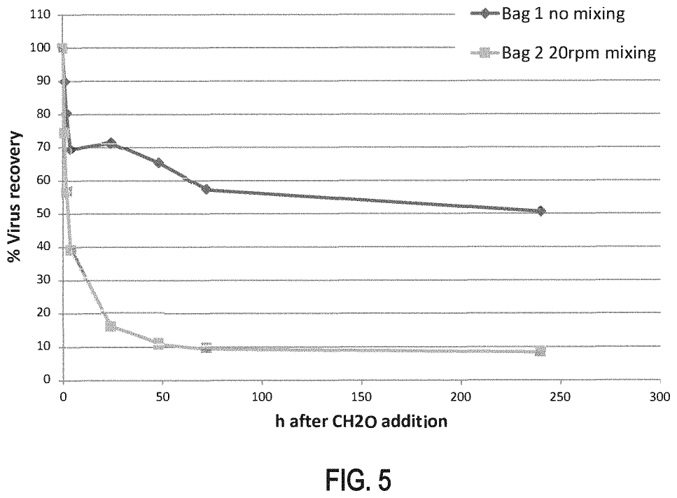

When comparing the virus recoveries after 10 days a nearly 5-fold increase in yield was achieved in bag #1 compared to the process mimicking bag #2 with an overall recovery of 51% without agitation and only 8% with constant agitation (FIG. 5). When assuming that the unspecific binding of virus was identical in both bags (as it should have been) the virus loss resulting from agitation would amount to ˜62% of total loss due to precipitation.

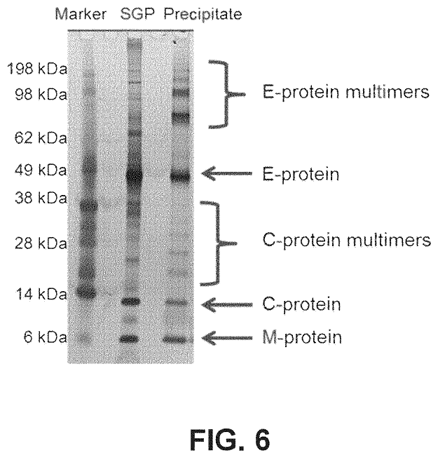

The formed precipitate was collected by centrifugation of the neutralized sample from bag #2, washed twice with PBS buffer and re-suspended in LDS buffer. Dissolved precipitate was analyzed on an SDS-PAGE/silver stain using SGP as comparison (FIG. 6). A number of additional bands could be observed, products of the formaldehyde cross linking reaction of both the viral E and C proteins. All three structural proteins of JEV (M, C, E) are clearly visible showing that the white precipitate indeed consist of aggregated virus particles.

In summary it could be clearly shown that the formation of precipitate during JEV production is correlated with the constant mechanical stress applied by agitation on the see-saw rocker during inactivation. By removing this stress the recovery of virus in our small-scale model could be increased by a factor of 5.

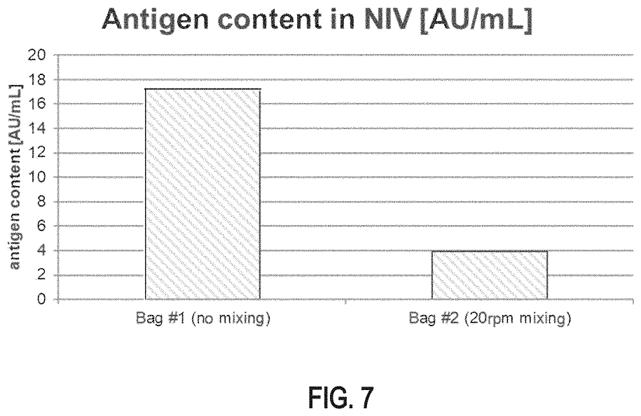

In order to evaluate if the difference in virus recovery seen in SE-HPLC can be confirmed by specific antigen content an inactivated JEV-ELISA was run on the day 10 NIV samples of both bags (FIG. 7). In good correlation to the HPLC results, the antigen content determined for bag #2 was only about 23% compared to bag #1.

### Example 3: Influence of Mixing Speed on Virus Recovery

To further evaluate the influence of constant mechanical stress on virus precipitation JEV was inactivated in Flexboy® bags using variable rocking speeds ranging from 0 (just inverting) to 20 rpm. In addition one bag of IVS was mixed for 10 min at 10 rpm immediately after filling and then stored without further mixing to evaluate how a short-term mixing at the beginning of the inactivation period affects virus recovery.

JEV SGP from lot JEV17D54 bottle B10 was diluted to 10 mAU*min using PBS buffer in a 1 L PETG bottle. 200 ppm formaldehyde was added to the bottle and the IVS was mixed for 3 min at 100 rpm. 130 mL IVS was transferred to each 500 mL bag followed by a 60 mL air cushion.

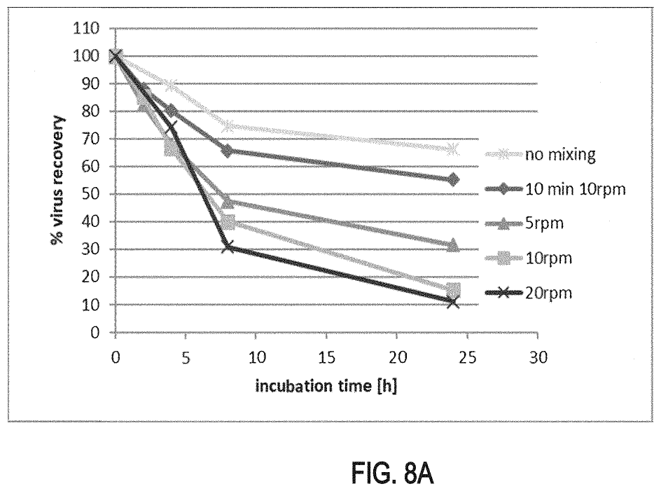

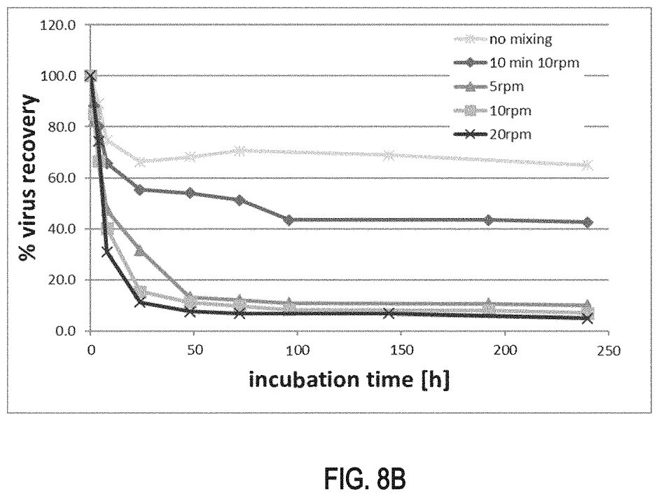

Bags were incubated at RT either on see-saw rockers in the dark or in a shaded box located next to the rockers. SE-HPLC analysis of virus peak recovery showed that regardless of the rocking speed the overall yield was only˜10% after 10 days (FIG. 8A). In comparison the virus recovery in the bag without mixing (just inverting) was 65% or more than 6-fold higher. However, when comparing only the first 24 h of inactivation a small difference in the kinetic can be seen for the different mixing speeds (FIG. 8B). After 8 h the recovery at 20 rpm was 10% lower than at 10 rpm and 20% lower than with 5 rpm. 24 h of constant mixing resulted in only 10% recovery at 20 rpm, ˜15% at 10 rpm but still 30% at 5 rpm indicating a direct correlation of mixing speed and recovery during the important first 24 h of inactivation.

Interestingly, the short initial mixing period of 10 min at 10 rpm did have a negative impact on virus recovery as the final yield was only˜43% (but still a 4-fold increase compared to the constantly mixed bags). In line with the better yield the amount of precipitate was also lower for this bag.

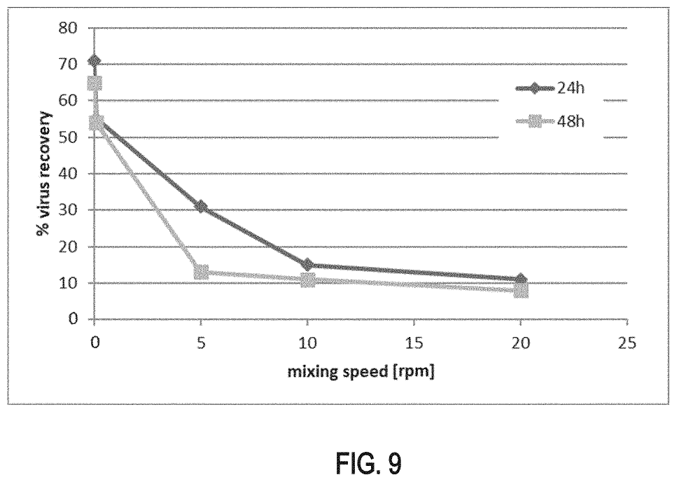

The observed correlation of mixing intensity and virus recovery is shown in FIG. 9.

In summary a correlation of rocking speed (=amount of mechanical stress) and virus recovery could be shown. However, even low rocking speeds resulted in final virus yields of ˜10% indicating a simple reduction in speed cannot be used to increase virus yields. In contrast, a short mixing pulse at the start of inactivation results in smaller losses and consequently higher yields. Such a short initial mixing step can be used in a virus inactivation during production where a thorough mixing is required while still reducing the mechanical stress to a bare minimum.

### Example 4: Addition of Formaldehyde Using Variable Mixing Speed in Flexboy® Bags

In the production process formaldehyde addition is done directly into the bag rather than before in a bottle followed by a short 10 min mixing at 30 rpm to assure homogeneous distribution.

To evaluate the effect of formaldehyde addition in bags followed by short mixing of the bag to formaldehyde addition and mixing in polycarbonate (PC) bottle diluted SGP from production lot JEV17K60 (stored in a 5 L Flexboy® bag) was inactivated. In total 4 bags were used for this experiment:
Bag #1: 22° C., CH2O addition in PC bottle, rocking of bag @ 30 rpm/10° angle/10 min;
Bag #2: 22° C., CH2O addition in PC bottle, no rocking of bag;
Bag #3: 22° C., CH2O addition in bag, rocking of bag @ 6 rpm/10° angle/10 min;
Bag #3: 22° C., CH2O addition in bag, rocking of bag @ 30 rpm/10° angle/10 min.

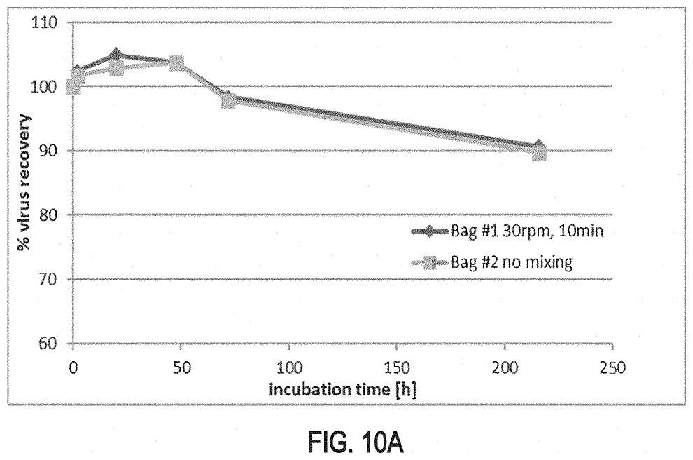

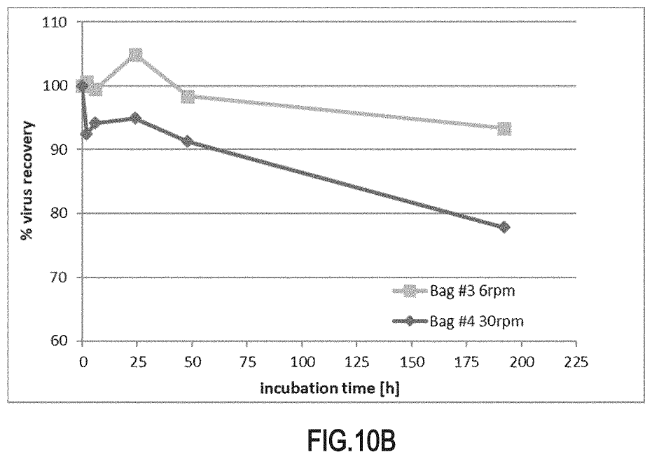

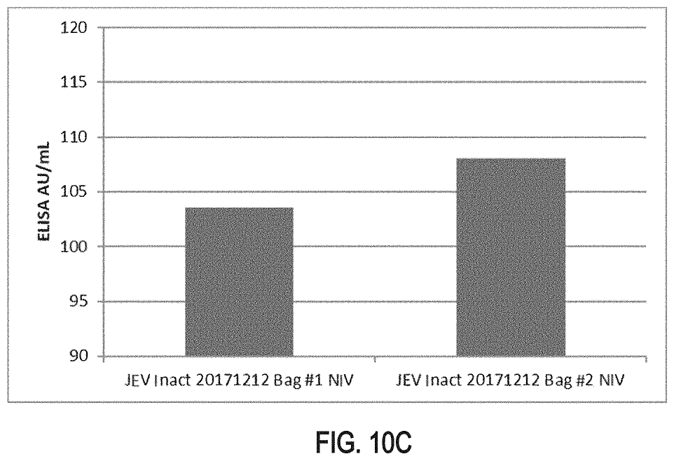

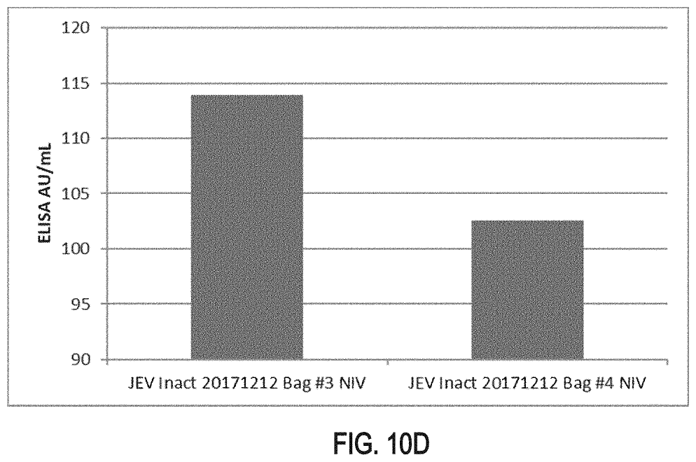

Virus recovery during inactivation was monitored by SE-HPLC analysis of virus peak area. In contrast to previous experiments where the starting material was frozen material from single sucrose gradient fractions the starting material for this experiment was diluted sucrose gradient pool from routine production stored only at 2-8° C. Using this material the overall virus yield after neutralization was higher with up to 90% recovery (FIG. 10A) compared to previous experiments and no influence of the short initial mixing step on virus recovery was observed.

Correspondingly, the specific antigen content after neutralization measured by inactivated JEV-ELISA was nearly identical for both bags (FIG. 10C).

The results on virus recovery for bags with formaldehyde addition and mixing in the bag are shown in FIG. 10B for virus peak area and FIG. 10D for inactivated JEV ELISA. When using a mild mixing step (bag #3, 6 rpm) the recovery after 10 days was identical to bags #1 and #2. This result indicates that for the initial step of formaldehyde addition and mixing it is irrelevant if the addition is done in the bottle or directly into the bag.

When using a harsh mixing step (bag #4, 30 rpm) the virus recovery is ˜10% lower in both virus peak area (FIG. 10B) and ELISA antigen content (FIG. 10D).

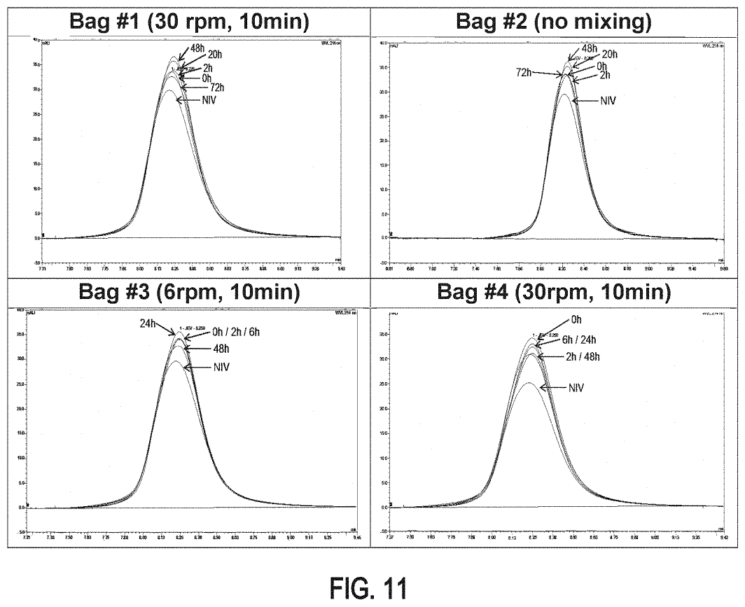

Overlays of the SE-HPLC chromatograms for each bag are shown in FIG. 11.

In summary, the method of formaldehyde addition had no impact on the virus yield after 10 days. Secondly, the mixing after formaldehyde addition should be done as gently as possible to further reduce mechanical stress and increase product recovery.

### Example 5: Evaluation of Flexsafe® Bags on JEV Inactivation Recovery

Flexsafe® bags are a new product line from Sartorius that feature a different inner surface layer. In the currently used Flexboy® bags the product contact layer is made from Ethylenvinylacetate (EVA). During γ-sterilization acetic acid is produced in detectable amounts. This can result in pH drops of the filled product. For example the pH of PBS buffer drops from 7.21 to 7.09 within 24 h of incubation when using a 500 mL Flexboy® bag filled with 130 mL buffer. This drop in pH can be even more pronounced when the surface to volume ratio is changed, e.g. by using 25 mL bags.

The new Flexsafe® bags contain an inner surface layer made from linear low density Polyethylen (LLDPE) that should not have this chemical side effect. Indeed, pH analysis during an inactivation experiment showed no effect of the bags on the sample pH.

However, the new bag design results in a different surface to volume ratio. A comparison of Flexboy® to Flexsafe® bags is shown in Table 2.

TABLE 2
Comparison of 500 mL Flexboy ® and Flexsafe ® bags

| | Flexboy ® bag | Flexsafe ® bag |
|---|---|---|
| Inner surface layer | Ethylenvinylacetate (EVA) | Liner Low Density |
| | | Polyethylen (LLDPE) |
| Filling volume | 500 mL | 500 mL |
| Film surface area | 452 cm² | 660 cm² |
| Dimension (L × W) | 184 mm × 120 mm | 240 mm × 130 mm |
| Surface to Volume ratio | 3.48 cm³/mL | 5.08 cm³/mL |
| (130 mL filling volume) | | |

Because of the different surface to volume ratio by using the same sample volume of 130 mL resulted in a 1.46× higher ratio compared to the Flexboy® bag model. Higher unspecific losses of virus due to adsorption to the bags surface can therefore influence the overall inactivation yields.

In total two Flexsafe® bags were incubated side-by-side containing 130 mL diluted SGP from Lot JEV17K60 and a 60 mL air cushion after addition of formaldehyde directly into the bag followed by an initial mixing step:
Bag #1: 22° C., rocking of bag @ 30 rpm/10° angle/10 min;
Bag #2: 22° C., rocking of bag @ 6 rpm/10° angle/10 min.

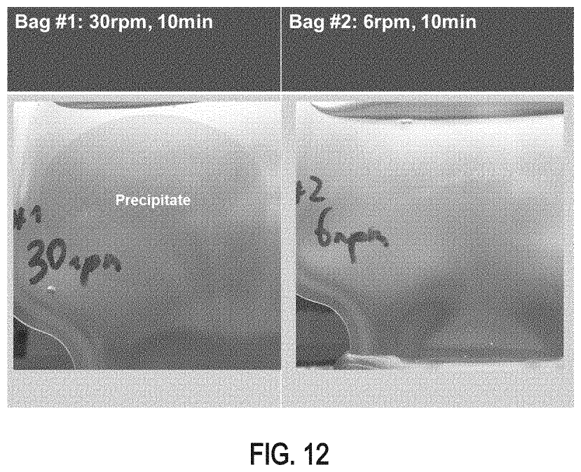

Virus recovery during inactivation was monitored by SE-HPLC. For Bag #1 (30 rpm mixing) some white precipitate was after 9 days as can be seen in FIG. 12; left panel, whereas no precipitate was observed for the gently mixed bag #2 (FIG. 12; right panel).

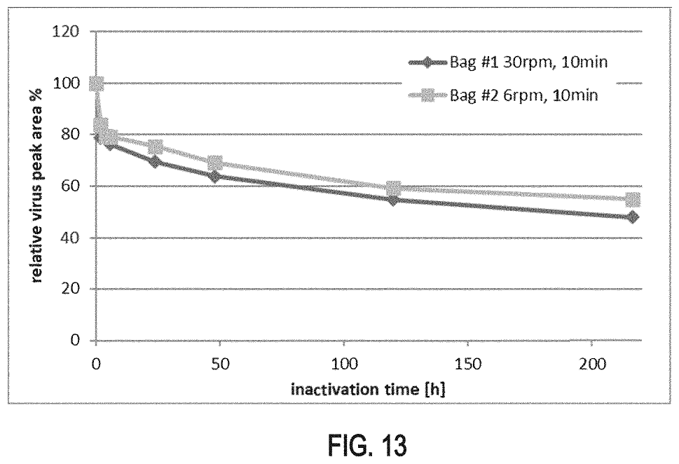

As shown in FIG. 13, despite the observed precipitation the overall recovery of virus after neutralization was only ˜10% lower in bag #1 (48% total recovery) compared to bag #2 (55% total recovery).

### Example 6: Comparison of Flexboy® and Flexsafe® Bags

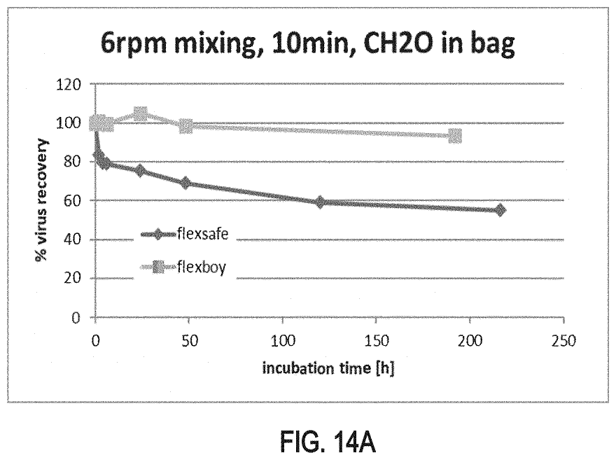

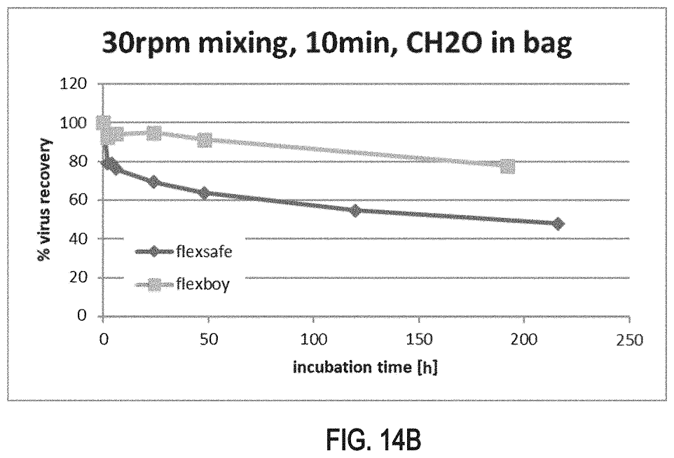

As already mentioned, the surface to volume ratio is significantly different between Flexboy® and Flexsafe® bags of the same nominal size. When comparing the results obtained for bags mixed at 6 rpm using a Flexsafe® bag the overall recovery was 38% lower (FIG. 14A). When using a harsh 30 rpm mixing step the difference was similar with 30% less recovery for the Flexsafe® bag (FIG. 14B).

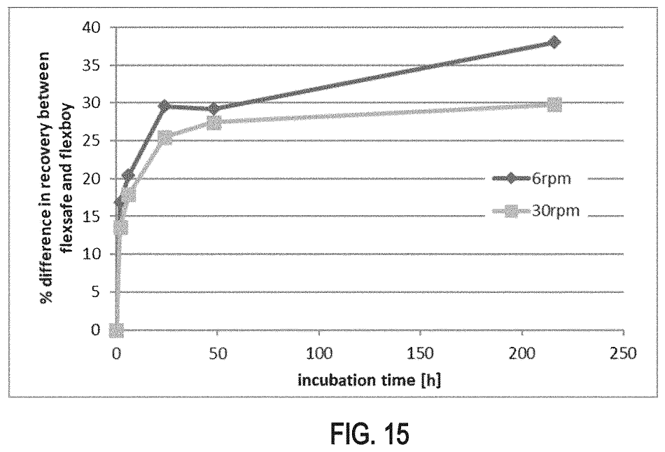

The difference in recovery between the two bag types is mainly during the first 24 h of inactivation with 25-30% higher losses in Flexsafe® than Flexboy® bags (FIG. 15) after which the formaldehyde reaction and unspecific adsorption to the bag surface are finished. Consequently, for the remaining 8 days of incubation the difference in recovery does not change dramatically indicating that both reactions have finished.

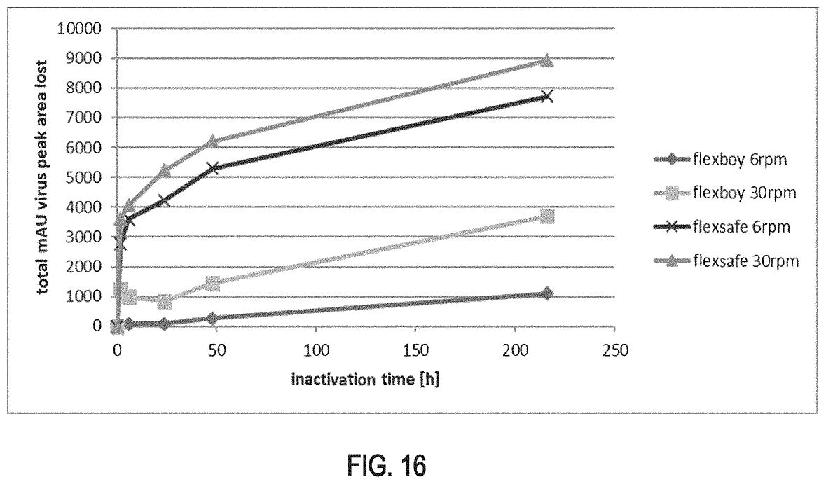

This two-phase reaction can be seen when plotting the total amount of virus loss (expressed as mAU virus peak area) during the inactivation (FIG. 16). For both Flexsafe® bags more than 50% of total virus losses occurred within the first 24 h of inactivation and ˜70% after 48 h. In contrast for the Flexboy® bag mixed with 6 rpm, the losses after 24 h were ˜8% and after 48 h ˜25% of the total virus loss. For the 30 rpm mixing<the initial losses were slightly bigger with ˜40% after 48 h which correlates with the observed virus precipitation in this bag.

When assuming the loss of virus during the first 48 h of inactivation for the 6 rpm mixing speed in both bag types is due to unspecific adsorption to the surface (no precipitation) the following losses per cm² bag surface can be calculated.

For the Flexboy® bag the total virus loss after 48 h was 273 mAU corresponding to 0.6 mAU per cm2 bag surface.

For the Flexsafe® bag the total virus loss after 48 h was 5300 mAU corresponding to 8.0 mAU per cm2 bag surface.

Taking into account the less favorable surface to volume ratio of the Flexsafe® bag (1.46) the expected unspecific losses would have been only 400 mAU indicating that the different inner surface layer of LLDPE seems to bind more JEV particles than the EVA membrane of the Flexboy® bags.

Concerning the degree of precipitation occurring in bags mixed with 30 rpm during the first 48 h of inactivation:

When assuming identical unspecific adsorption in both cases (6 rpm or 30 rpm) the additional losses for both Flexsafe® and Flexboy® bag are nearly identical at 5-7% of starting virus indicating that roughly the same amount of virus is lost due to precipitation regardless of the bag geometry or chemical composition.

### Example 7: Change in Virus Inactivation when Using a 37° C. Incubation Step

Inactivation by formaldehyde is influenced by the reaction temperature. Inactivation at higher temperatures (e.g. 37° C.) during the first 24 h is used in the production of tick-borne encephalitis (TBE) vaccine. Afterwards the temperature is lowered to 22° C. for the remaining incubation time. A similar approach used for JEV could possibly result in faster inactivation and reduced number of hold days after no infectious particle are detected.

To test this hypothesis two 500 mL Flexboy® bags were filled with 130 mL diluted SGP from lot JEV16G35 bottle B16 and incubated as follows:
Bag #1: 37° C. for 24 h, then 22° C. without rocking (just inverted);
Bag #2: 37° C. for 24 h, then 22° C. on a see-saw rocker at 20 rpm, 8° angle.

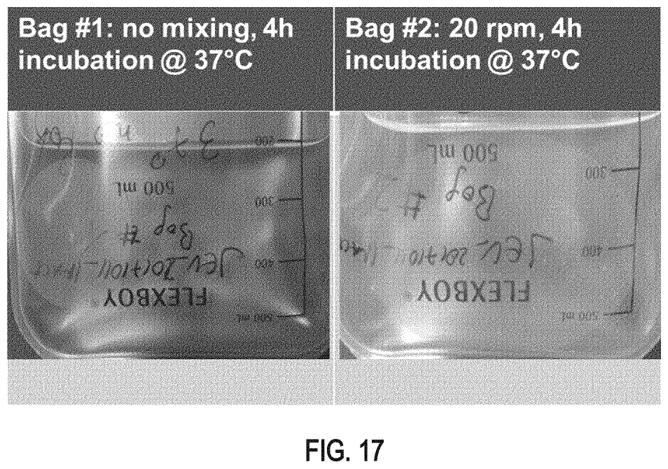

Bags were inspected visually every hour during the first 8 h of inactivation. Already after 2 h of incubation severe precipitation was observed for the bag mixed at 20 rpm and significant precipitation for the bag without mixing (FIG. 17). This result indicates that JEV cannot be inactivated at higher temperatures without the induction of significant precipitation.

When comparing the virus recovery by SE-HPLC of the two 37° C. bags to reference bags inactivated at room temperature after 24 h the dramatic impact of higher temperatures on JEV recovery can be seen even better.

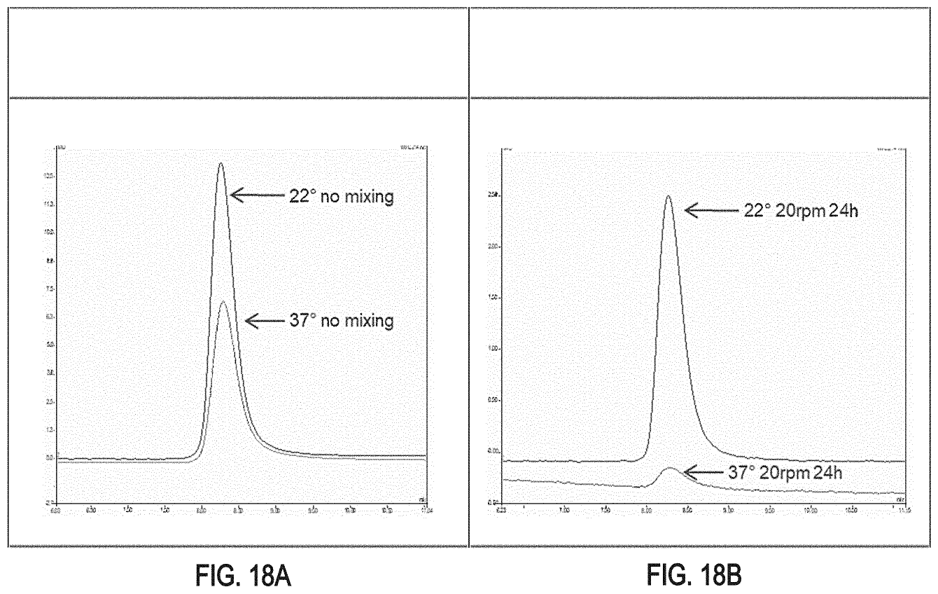

Without any mixing the recovery of virus particles was only 58% for the 37° C. incubation compared to the reference bag (FIG. 18A). With constant mixing the difference was even more pronounced with only 7% recovery (FIG. 18B).

After 24 h (when the bags were transferred to 22° C.) in the constantly mixed bag nearly no peak could be detected by SE-HPLC anymore indicating complete loss of virus

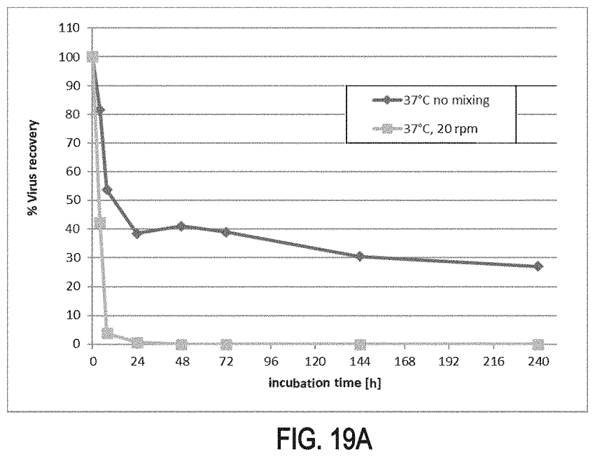

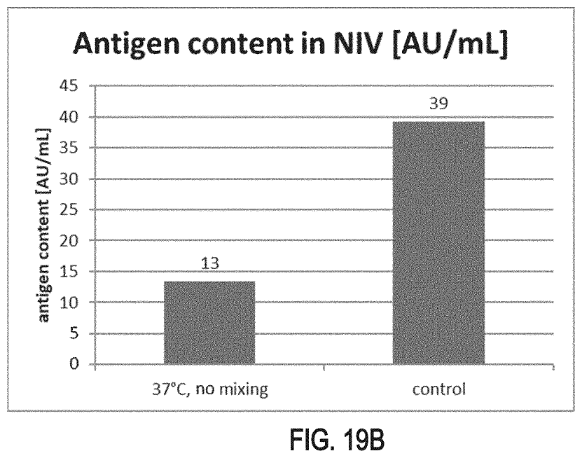

(FIG. 19A). Without mixing the overall yield by SE-HPLC was slightly better at ˜27% but less than half of the control bag incubated at 22° C. Antigen content determined by inactivated JEV ELISA showed the same results (FIG. 19B). When incubated at 37° C. without mixing the antigen recovery is only ˜34% compared to a control bag. When the bag was constantly mixed no ELISA signal could be detected at all confirming the SEC data of complete virus loss.

Taken together, these results indicate that JEV cannot be inactivated using a 37° C. high temperature step as used for TBE virus inactivation.

### Example 8: Influence of Mixing Speed on Virus Stability Using Polycarbonate Bottles

In the production process, a sucrose gradient pool is collected in PC bottles and mixed extensively during dilution using magnetic stirrer at high speed (430 rpm). To analyze the effect of mixing on infectious virus particles, a mixing study at small-scale was conducted.

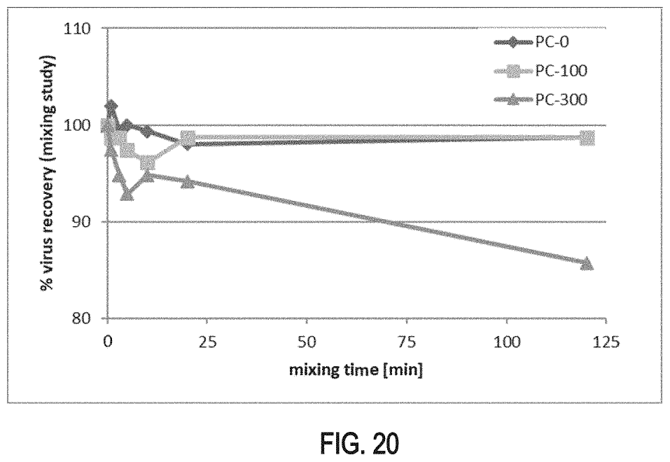

50 mL of diluted SGP from lot JEV17K60 was stirred in 125 mL PC bottles at 0 (just swirling by hand), 100 and 300 rpm for 120 min in total. Samples were drawn after 0, 1, 3, 5, 10, 20, and 120 min of constant mixing and analyzed by SE-HPLC. While mild mixing conditions up to 100 rpm did not result in virus loss, the recovery did drop by 14% when using harsh mixing conditions (FIG. 20; formation of a strong vortex was observed at 300 rpm mixing speed).

After 120 min of mixing the samples were stored at 22° C. overnight without further disturbance. No change in virus recovery was observed during this time.

To test if the different amount of mechanical stress before formaldehyde addition results in differences during inactivation, all three samples were incubated for 10 days at 22° C. in the dark after formaldehyde addition. Immediately after formaldehyde addition each sample was mixed for 10 min using the same mixing speed as before.

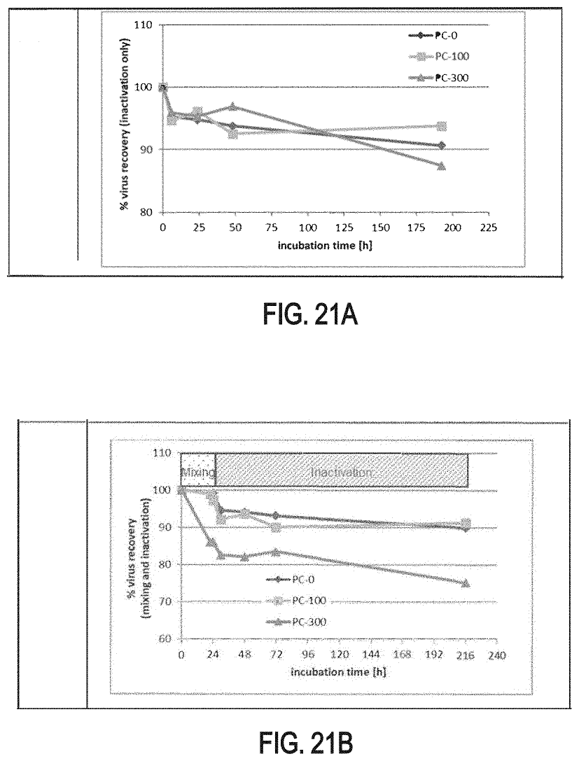

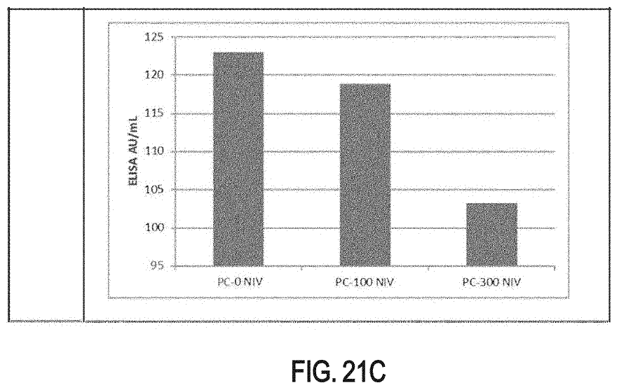

SE-HPLC analysis of virus recovery showed very high yields after neutralization and no effect of different mixing speeds on JEV yields during inactivation (FIG. 21A). When including the losses during the initial mixing the overall recovery of JEV when using harsh mixing conditions was ˜15% lower compared to the mild conditions (FIG. 21B).

NIV samples were analyzed by inactivated JEV ELISA showed the same reduced antigen content for the PC-300 sample as seen with SE-HPLC (FIG. 21C).

Taken together, extensive mixing using harsh conditions (high speed, vortex formation) have a negative impact on the overall inactivation yield for JEV.

### Example 9: Correlation of Reynolds Number and Virus Recovery

A modified Reynolds number (Re_{mod}) was introduced to describe fluid flow in wave bioreactors (Eibl et al., 2009). This dimensionless number describes the ratio of internal force to internal friction. The Reynolds number is generally governed by equation 1, where w is the fluid velocity, l is the characteristic length of the system (in our case, of the flexible bioreactor bag), and v is the kinematic viscosity of the culture medium.

$$Re = \frac{w * l}{v} \qquad Equation 1$$

The modified Re number (see Equation 2) can be used to describe and characterize fluid flow in flexible bioreactor bags.

$$R {e}_{mo d} = \frac{V * k * C * D}{15 * v * ( 2 * h + B )} \qquad Equation 2$$

V=working volume,
k=rocking rate,
C and D=empirical constant; differs for every bag type, rocking rate, rocking angle and culture volume,
h=liquid level (height),
B=width of bag,
v=kinematic viscosity of the medium.

Usually, a transition range from laminar to turbulent flow was determined ranging from Re_{mod} between 200 and 1000. These transition areas vary according to the type of flexible bioreactor bag used.

In this example JEV inactivation is done in a 20 L bag with an average fill volume of approx. 12.5±2.5 L. This variable process volume is caused by dilution of sucrose gradient purified JEV to a target total protein content of 50 μg/ml at start of inactivation. This variable inactivation volume results in a quite broad range of liquid level h in the bag. According to equation 2 one would expect higher Re_{mod} at lower liquid level h in the same bag geometry. Hence, a higher Re_{mod} would indicate more intensive mixing and air-liquid interface formation and finally lower process yield due to virus particle precipitation.

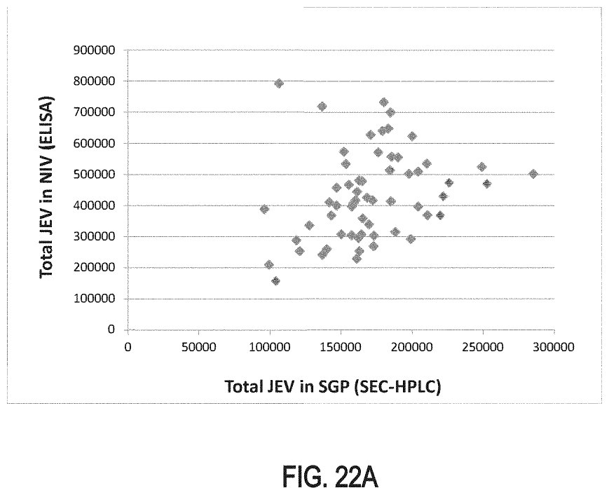

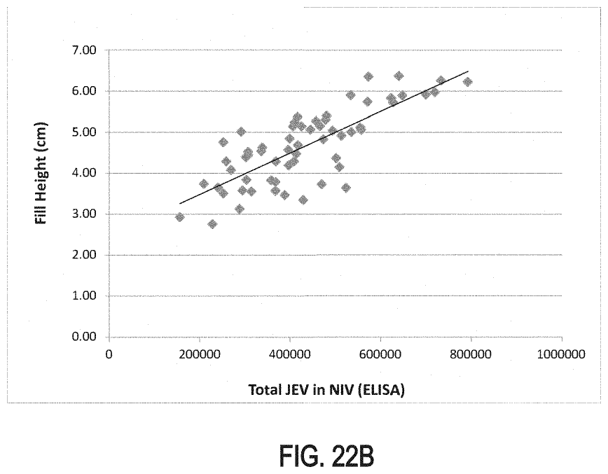

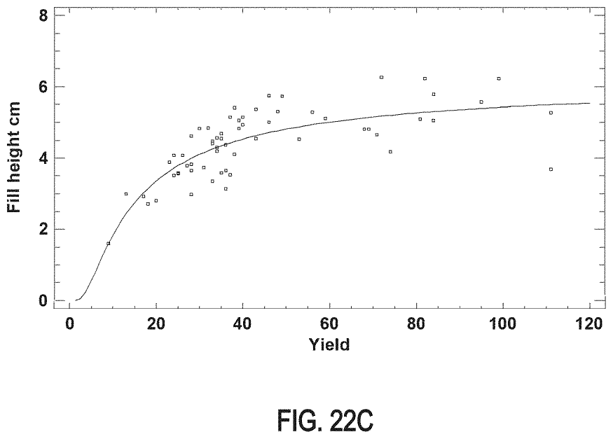

Correlation analysis of recovered JEV in NIV vs. fill height in 20 L bag (equivalent to inactivation volume) exactly follows that prediction (see FIG. 22). JEV recovery increases at higher filling volume of the bag because wave formation and mixing effects are less pronounced.

In this case the following parameters were determined: n=61 lots, 20 L bags, 8 rpm, 10° rocking angle, density 1 kg/dm³, kinematic viscosity 0.00000103 m²/sec. Corresponding fill height was calculated depending on the bag fill volume.

The correlation factor C (see equation 2) was taken from Table 3 of Eibl & Eibl (2006) (extrapolated for each fill height), and D was constant and equal to 0.312 for 20 L bag as indicated in Table 4 of Eibl & Eibl (2006).

TABLE 5
Correlation between Re_{mod} and virus yield.

| | | | | C | Re |
|---|---|---|---|---|---|
| Yield (%) | Fill height (cm) | V (L) | Fill level (%) | (extrapolated) | mod |
| 13 | 3 | 8.5 | 43 | 0.68 | 1314 |
| 20 | 3.2 | 9.5 | 48 | 0.6 | 1283 |
| 33 | 4.2 | 12.5 | 63 | 0.45 | 1229 |
| 39 | 4.8 | 13.5 | 68 | 0.4 | 1168 |
| 47 | 5.2 | 14.5 | 73 | 0.35 | 1088 |
| 66 | 5.2 | 14.5 | 73 | 0.35 | 1088 |
| 82 | 5.4 | 15 | 75 | 0.3 | 960 |

‘C values extrapolated from Table 3 (p. 212) published in Eibl & Eibl (2006).

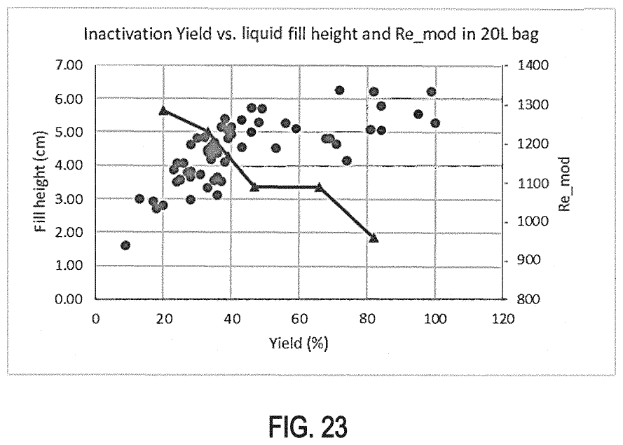

It can be seen from FIG. 23 that Re_{mod} decreases with increased filling level in 20 L Wave Bag working with higher volume. Increased filling level results in reduced headspace volume, so that the linear development of the wave movement is no longer possible after a certain point (Re-critical). This effect minimizes the gas-liquid interface mixing effects causing virus particle precipitation and helps to increase virus recovery (yield) during 10 days inactivation time. In this case the Re-critical is estimated in the in the range of Re_{mod}˜1000.

### Summary

In the current approved IXIARO® manufacturing process the virus inactivation step yields only about 34%. This means that 2/3 of the product is lost in each production lot at this step. Consequently, reducing losses during this step will immediately impact the overall productivity of the process significantly. Using a scale-down model it could be shown that the step yield for inactivation can be dramatically improved by a simple process change, reducing the mixing during inactivation to a bare minimum. JEV, as other small RNA viruses like ZIKV, is highly susceptible to mechanical stresses like high speed mixing on magnetic stirrers and harsh continuous mixing on see-saw rockers.

In our small-scale model the step yield for JEV could be increased by a factor of 4-6 as minimized mechanical stress resulted in recoveries of up to and above 90% while mixing at standard manufacturing process speeds resulted in recoveries below 10%.

The surface to volume ratio in our model was 10 times less favorable compared to production scale resulting in much higher losses to unspecific adsorption. It can therefore be concluded that the yield increase during routine production could be up to 2.5-3 fold compared to the current process. This would result in more than a doubling of the annual production without significantly changing the process.

### Example 10: Virus Inactivation Process with CHIKV Using Formaldehyde

Sucrose gradient pool from a production lot was diluted with PBS buffer in a 500 mL PETG bottle and mixed for 3 minutes. SE-HPLC showed the diluted SGP had a virus peak area of 1.8 mAU*min. 130 mL of diluted SGP were transferred to three 500 mL Flexboy® bags and a 60 mL air cushion was added to each bag using a syringe. Formaldehyde was added directly to the bags using a syringe (200 ppm final concentration):

Bag #1: 22° C., CH2O addition in bag, rocking of bag @ 6 rpm/10° angle

Bag #2: 22° C., CH2O addition in bag, rocking of bag @ 30 rpm/10° angle

Bag #3: 22° C., CH2O addition in bag, rocking of bag @ 6 rpm/10° angle/10 min

Virus recovery during inactivation was monitored by SE-HPLC analysis of virus peak area. For each bag, a ˜four mL sample was drawn using a syringe at various time points within 9 days. The sample was neutralized immediately by addition of 4 mM sodium metabisulfite and incubation for 3 min at RT. Samples for SE-HPLC analysis were stabilized by addition of 50 μg/mL BSA and analysed immediately. For TCID50 analysis samples were stabilized with 50% fetal bovine serum and stored frozen at <−70° C. until analysis. Retain samples were supplemented with 1/10 volume of 10× stabilization buffer (50% glycerol, 500 μg/mL BSA in PBS) and stored frozen at <−70° C.

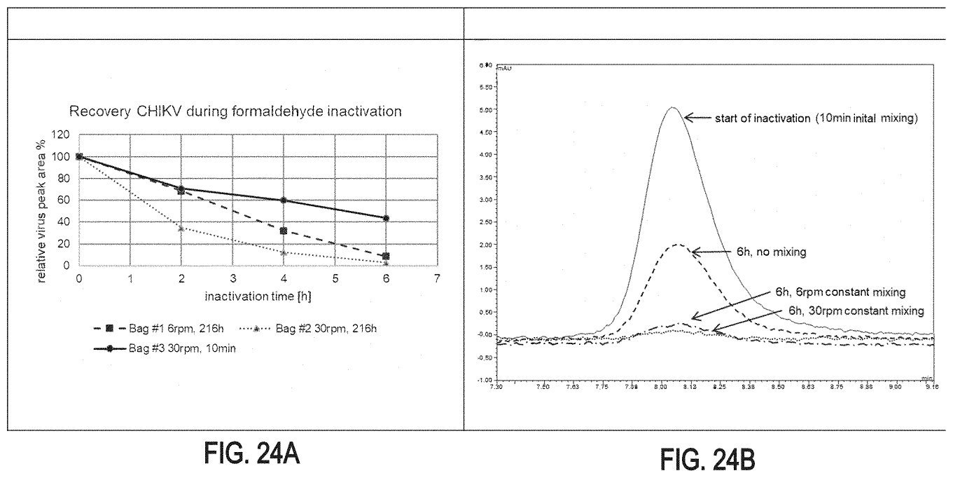

Immediately after start of inactivation, a significant impact of constant agitation on virus recovery was observed as the observed virus peak decreased by more than 60% for the 30 rpm mixing and only 30% for the 6 rpm mixed bag (FIG. 24A). Recovery for the 30 rpm bag dropped to 3% after just 6 h and 9% for the bag mixed at constantly 6 rpm. For both of these bags no virus signal was observed after 48 h of inactivation (when the 0.2 μm filtration step into a new bag would normally be conducted). Therefore, the experiments were terminated after 48 h.

The virus recover in Bag #3 (only initial 10 min mixing, then no agitation at all) however remained relatively constant after the initial drop at 33% after 24 h and 22% after 48 h.

Overlays of the SE-HPLC results for the three bags after 6 h of inactivation and the starting material are shown in FIG. 24B. Whereas the constantly mixed bags showed nearly no virus peak signal any more a significant amount was still detectable in the non-agitated bag #3.

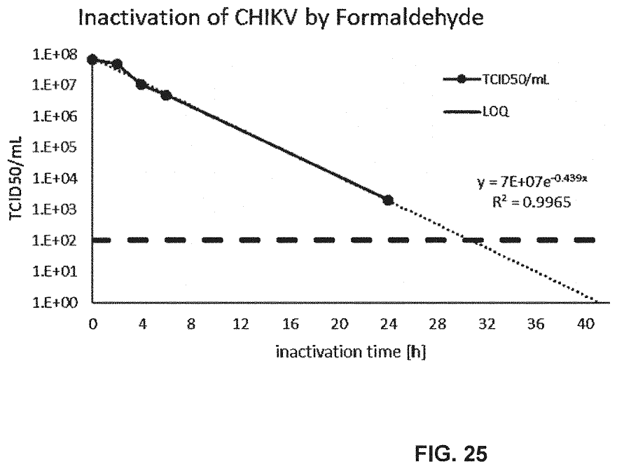

TCID50 analysis of samples taken during the first 48 h showed a fast inactivation of Chikungunya virus by formaldehyde with a 99% reduction after ˜9 h and a 99.9% reduction after ˜15 h. Virus titer was below the limit of quantification within after ˜30 h and complete inactivation was achieved after ˜41 h (FIG. 25) based on regression analysis.

In conclusion, similar to the inactivation of JEV the virus recovery during inactivation for Chikungunya virus is influenced by mechanical stress. Reducing mechanical stress can therefore improve the overall yield of virus during inactivation.

### REFERENCES

Metz et al., Identification of formaldehyde-induced modifications in proteins: reactions with model peptides. J Biol Chem. 2004 Feb. 20; 279(8):6235-43.
Kiernan. Formaldehyde, formalin, paraformaldehyde and glutaraldehyde: What they are and what they do. Microscopy Today. 2000; 00-1: 8-12.
Eibl et al. Bag bioreactor based on wave-induced motion: characteristics and applications. Adv Biochem Eng Biotechnol. 2009; 115:55-87.
Eibl & Eibl. Design and use of the Wave Bioreactor for plant cell culture. In: Dutta Gupta S, Ibaraki Y (eds) Plant tissue culture engineering, series: focus on biotechnology, vol 6. Springer, Dordrecht, 2006, pp. 203-227.
Maa and Hsu. Protein denaturation by combined effect of shear and air-liquid interface. Biotechnol Bioeng. 1997 Jun. 20; 54(6):503-12.

1. A method of inactivating a virus comprising
contacting a liquid composition comprising the virus with a chemical viral inactivating agent in a container,
mixing the chemical viral inactivating agent and the liquid composition comprising the virus under conditions of laminar flow but not turbulent flow, and
incubating the chemical viral inactivating agent and the liquid composition comprising the virus for a time sufficient to inactivate the virus.

2. The method of claim 1, wherein mixing of the chemical viral inactivating agent and the liquid composition comprising the virus is performed in a flexible bioreactor bag.

3. The method of claim 2, wherein the mixing is performed under conditions that result in a modified Reynolds Number (Re_{mod}) of less than 1000, as determined by formula (1):
$$R {e}_{mo d} = \frac{V * k * C * D}{15 * v * ( 2 * h + B )} , \qquad ( 1 )$$
wherein V is the volume of the flexible bioreactor bag, k is the mixing rate of the flexible bioreactor bag, C and D are correlation factors determined for the flexible bioreactor bag, v is the kinematic viscosity of the liquid in the flexible bioreactor bag, h is the height of liquid in flexible bioreactor bag, and B is the width of the flexible bioreactor bag.

4. The method of claim 1, wherein the mixing comprises inverting the container not more than 1, 2, 3, 4 or 5 times during the period of incubation.

5. The method of claim 1, wherein the mixing comprises subjecting the container to rocking, rotation, orbital shaking, or oscillation for not more than 15 seconds, 30 seconds, 1 minute, 2 minutes, 5 minutes, or 10 minutes at not more than 2 rpm, 5 rpm, or 10 rpm, during the period of incubation.

6. The method of claim 1, wherein the mixing is performed only within the first 10 minutes, 20 minutes, 30 minutes, 40 minutes, 50 minutes, or 60 minutes after the contacting of the virus and the agent in the container, or wherein no mixing is performed after 2 hours, 4 hours, 8 hours, 12 hours, 24 hours, or 48 hours from the contacting of the virus and the agent in the container.

7. The method of claim 1, wherein the inactivation of the virus is completed in a time period that is not more than 10% longer than the time period for inactivation of the same virus using the same chemical viral inactivation agent without any restriction on mixing.

8. The method of claim 1, wherein the chemical viral inactivation agent comprises or consists of formaldehyde; enzyme; β-propiolactone;
ethanol; trifluroacetic acid; acetonitrile; bleach; urea; guanidine hydrochloride; tri-n-butyl phosphate; ethylene-imine or a derivative thereof; an organic solvent, optionally Tween, Triton, sodium deoxycholate, or sulfobetaine; or a combination thereof.

9. The method of claim 1, wherein the chemical viral inactivating agent and the liquid composition comprising the virus are incubated for 1-20 days.

10. The vaccine of claim 1, wherein the chemical viral inactivating agent and the liquid composition comprising the virus are incubated at about 10° C. to about 30° C.

11. The method of claim 1, wherein the virus is a RNA virus.

12. The method of claim 11, wherein the RNA virus belongs to a virus family selected from the group consisting of Flaviviridae, Togaviridae, Paramyxoviridae, Picornaviridae, Orthomyxoviridae, Filoviridae, Arenaviridae, Rhabdoviridae, and Coronaviridae.

13. The method of claim 12, wherein the virus is selected from the group consisting of Japanese encephalitis virus, Zika virus, Yellow Fever virus, Dengue virus, thick born encephalitis virus, polio virus, hepatitis A virus, rabies virus, hepatitis B virus, hepatitis C virus and Chikungunya virus.

14. The method of claim 1, wherein the liquid composition comprising the virus comprises a sucrose gradient pool of purified virus.

15. The method of claim 1, wherein the volume of the liquid composition comprising the virus and the chemical viral inactivating agent in the container is within 10%, 5%, 2%, or 1% of the volume calculated to provide the minimum gas-liquid interface size for the container.

16. The method of claim 1, wherein the volume of the liquid composition comprising the virus and the chemical viral inactivating agent in the container is within 10%, 5%, 2%, or 1% of the maximum volume recommended by the manufacturer of the container.

17. The method of claim 1, wherein an interior surface of the container comprises ethylenvinylacetate (EVA).

18. The method of claim 1, wherein the mixing under conditions of laminar flow but not turbulent flow results in a recovery of virus that is at least 20% more than the recovery of virus under standard mixing conditions.

19. An inactivated virus preparation produced by the method of claim 1.

20. The inactivated virus preparation of claim 19 for use in treating or preventing a viral infection.
